(19) United States
(12) Patent Application Publication
(10) Pub. No.: US 2026/0335626 A1
(43) Pub. Date: Oct. 29, 2026

(54) PARAMETER CALCULATION OF A BLOOD PUMP

(71) Applicant: BiVACOR Inc., Huntington Beach, CA (US)

(72) Inventors: Nicholas GREATREX, Huntington Beach, CA (US); Daniel TIMMS, Huntington Beach, CA (US)

(21) Appl. No.: 19/484,708

(22) PCT Filed: May 15, 2024
(86) PCT No.: PCT/US2024/029438

§ 371 Date: Nov. 14, 2025

Related U.S. Application Data
(60) Provisional application No. 63/502,505, filed on May 16, 2023.

(51) Int. Cl. A61M 60/178; A61M 60/816; A61M 60/822

(52) U.S. Cl. CPC A61M 60/178; A61M 60/816; A61M 60/822

(57) ABSTRACT

A heart pump including a housing forming a cavity including at least one inlet and at least one outlet, an impeller provided within the cavity, the impeller including vanes for urging fluid from the inlet to the outlet upon rotation of the impeller, a drive that rotates the impeller within the cavity and a magnetic bearing including at least one bearing coil that controls an axial position of the impeller within the cavity. One or more electronic processing devices are provided that are configured to determine a bearing indicator relating to axial forces on the impeller and use the bearing indicator to calculate at least one parameter.

### CROSS REFERENCE(S) TO RELATED APPLICATION(S)

This application claims priority to U.S. Provisional Application No. 63/502,505, filed on May 16, 2023, the entire contents of which are hereby incorporated by reference.

### BACKGROUND OF THE INVENTION

The present invention relates to a heart pump and in particular to a method of calculating parameters, such as subject parameters, hemodynamic parameters or pump operational parameters for a heart pump, and optionally using these to control operation of the pump.

### DESCRIPTION OF THE PRIOR ART

The reference in this specification to any prior publication (or information derived from it), or to any matter which is known, is not, and should not be taken as an acknowledgment or admission or any form of suggestion that the prior publication (or information derived from it) or known matter forms part of the common general knowledge in the field of endeavour to which this specification relates.

The use of rotary impeller based mechanical pumps to treat heart failure is increasing as the general population ages and the number of donor organs for heart transplantation remains limited. Devices can be used to bridge a patient to heart trans-plant, to recovery, or indeed as a destination alternative.

WO2004098677 and WO2006053384A1 each describe a double sided impeller that rotates at a common speed, with each side of the impeller respectively configured for left and right heart assistance. This effectively introduces an inherent problem regarding the ability to independently control and thus balance the outflow from the left and right sides of the device, i.e. an increase in impeller rotational speed with produce a corresponding increase in outflow from both cavities.

WO2006053384A1 addressed this issue by introducing the ability to axially displace the rotating impeller within the cavity so as to simultaneously alter the relative efficiencies of each side of the device. However, when the control method used to achieve this axial displacement is active, such pumps require the use of feedback signals from pressure sensors and the like to actively control and maintain a desired set axial location. This method of control would inherently consume excessive amounts of electrical power and introduce issues relating to the long term reliability of blood contacting sensors.

U.S. Pat. No. 8,636,638 describes a controller for a heart pump that determines movement of an impeller within a cavity in a first axial direction, the cavity including at least one inlet and at least one outlet, and the impeller including vanes for urging fluid from the inlet to the outlet, causing a magnetic bearing to move the impeller in a second axial direction opposite the first axial direction, the magnetic bearing including at least one coil for controlling an axial position of the impeller within the cavity, determining an indicator indicative of the power used by the magnetic bearing and causing the magnetic bearing to control the axial position of the impeller in accordance with the indicator to thereby control a fluid flow between the inlet and the outlet.

U.S. Pat. No. 7,435,059 describes a system for pumping blood to assist or assume the cardiac function of a patient is characterized by a blood pump that exhibits a steep pump curve such that only small changes in pump flow occur for large changes in differential pressure across the pump. The pump therefore exhibits flow-limiting characteristics to protect the physiological system against harmful flow rates. Pump flow may also be limited by controlling the current provided to a driver from a power supply or by suitable restrictions within or external to the pump housing.

WO2017120451 describes a heart pump including a housing forming a cavity including at least one inlet and at least one outlet, an impeller provided within the cavity, the impeller including vanes for urging fluid from the inlet to the outlet upon rotation of the impeller, a drive that rotates the impeller within the cavity, a magnetic bearing including at least one bearing coil that controls an axial position of the impeller within the cavity and a controller. The controller includes an electronic processing device that monitors changes in a bearing indicator in response to a perturbation in blood flow, the bearing indicator being at least partially indicative of operation of the magnetic bearing and controls the drive to thereby selectively change a rotational speed of the impeller at least partially in accordance with changes in the bearing indicator.

WO2017120453 describes a heart pump including a housing defining a cavity including at least one inlet and at least one outlet, an impeller provided within the cavity, the impeller including vanes for urging fluid from the inlet to the outlet upon rotation of the impeller, a drive that rotates the impeller within the cavity, a magnetic bearing including at least one bearing coil that controls an axial position of the impeller within the cavity, a sensor that senses an axial position of the impeller within the cavity and a controller. The controller includes an electronic processing device that, in response to a change in axial hydraulic forces on the impeller determines an axial position of the impeller within the cavity, determines a reference power in accordance with the determined axial position and controls the magnetic bearing to cause the impeller to move until a bearing power indicator indicative of the power used by the magnetic bearing reaches the reference power.

It is desirable to be able to control the rotational speed of impellers, in order to optimise flow for the particular physiological conditions of a subject. However, measuring hemodynamic parameters can be difficult, requiring sensors to be implanted within the subject.

U.S. Pat. No. 11,040,188 describes methods and apparatus for estimating flow rate in a blood circulation assist system employing impeller eccentricity. The method includes magnetically rotating an impeller within a blood flow channel of a blood pump. The impeller is levitated within the blood flow channel transverse to the impeller axis of rotation. A rotational speed for the impeller is determined. At least one impeller transverse position parameter is determined. The at least one impeller transverse position parameter is based on at least one of (1) an amount of a bearing current that is used to levitate the impeller transverse to the impeller axis of rotation, and (2) a position of the impeller within the blood flow channel transverse to the impeller axis of rotation. A flow rate of blood pumped by the blood pump is estimated based on the impeller rotational speed and the at least one impeller transverse position parameter.

This system uses lateral forces, perpendicular to the axis of rotation, to calculate a flow rate through the pump. This is of only limited accuracy and is unsuitable for dual sided pumps.

U.S. Pat. No. 6,527,699 describes a non-contact axial flow turbo blood pump for propelling blood, which is composed of a pump housing that defines a pump axis, with inlet, outlet openings at opposite axial ends of the pump housing, a rotor unit that defines a rotor axis, and opposing rotor axial ends. The pump magnetically suspends the rotor within the pump housing at the rotor axial ends so as to avoid causing physical contact between the housing to define fluid gaps between the rotor axial ends, and the magnetic suspension elements.

The document further describes that an axial bearing position is monitored by measuring the coil inductance to determine an associated force. A differential pressure DP is then calculated by dividing the derived force by the rotor area.

US20100174231 describes a method of controlling a pump including an electrical motor coupled to a rotor which carries first and second impellers at opposite ends thereof. The method includes: (a) driving the rotor using the motor, so as to circulate fluid from the first impeller through a first fluid circuit, the second impeller, a second fluid circuit, and back to the first impeller; (b) determining a resistance of the first fluid circuit, based on a first motor parameter; (c) determining a flow rate through the first fluid circuit based on a second motor parameter; and (d) varying at least one operational parameter of the pump so as to maintain a predetermined relationship between the flow rate and the resistance of the first fluid circuit.

Thus, this approach only uses the motor speed and power to calculate systemic and vascular resistances.

“Sensorless Viscosity Measurement in a Magnetically-Levitated Rotary Blood Pump” by Wataru Hijikata, Jun Rao, Shodai Abe, Setsuo Takatani, Tadahiko Shinshi 2015 describes controlling the flow rate in an implantable rotary blood pump based on the physiological demand made by the body. The approach uses a sensorless method for measuring viscosity in magnetically levitated rotary blood pumps, which requires no additional sensors or mechanisms. By applying vibrational excitation to the impeller using a magnetic bearing, the viscosity of the working fluid is determined by measuring the phase difference between the current in the magnetic bearing and the displacement of the impeller.

However, this approach only estimates fluid viscosity and hence haematocrit, but does not estimate pressure or flow.

### SUMMARY OF THE PRESENT INVENTION

In one broad form, an aspect of the present invention seeks to provide a heart pump including: a housing forming a cavity including at least one inlet and at least one outlet; an impeller provided within the cavity, the impeller including vanes for urging fluid from the inlet to the outlet upon rotation of the impeller; a drive that rotates the impeller within the cavity; a magnetic bearing including at least one bearing coil that controls an axial position of the impeller within the cavity; and, one or more electronic processing devices that are configured to: determine a bearing indicator relating to axial forces on the impeller; and, use the bearing indicator to calculate at least one parameter.

In one embodiment the bearing indicator is indicative of at least one of: a tilt of the impeller; an electrical current used by the magnetic bearing; an axial position of the impeller within the cavity; a change in tilt of the impeller; a rate of change in tilt of the impeller; a change in bearing indicator; a rate of change in bearing indicator; a derivative of bearing indicator; an integral of bearing indicator; a magnitude in difference between maximum and minimum values of bearing indicator over a pulse period; a change in electrical current used by the magnetic bearing; a rate of change in electrical current used by the magnetic bearing; a derivative of electrical current used by the magnetic bearing; an integral of electrical current used by the magnetic bearing; a magnitude in difference between maximum and minimum values of electrical current used by the magnetic bearing over a pulse period; a change in axial position of the impeller within the cavity; a measure of vibration of the impeller; and, a rate of change in axial position of the impeller within the cavity.

In one embodiment the one or more processing devices are configured to determine the bearing indicator based on at least one of: signals from a position sensor configured to detect an axial position of the impeller; signals from a position sensor configured to detect a radial position of the impeller; and, a current supplied to the at least one bearing coil.

In one embodiment the one or more electronic processing devices are configured to: determine a drive indicator indicative of rotation of the impeller; and, use the drive indicator and the bearing indicator to calculate the at least one parameter.

In one embodiment the drive indicator is indicative of at least one of: a current supplied to the drive; an expected rotational speed of the impeller; an actual rotational speed of the impeller; and, a magnitude of a rotational speed change of the impeller; a waveform of changes in rotational speed of the of the impeller; and, a ratio of high speed and low speed periods of the waveform.

In one embodiment the one or more processing devices are configured to determine the drive indicator based on a magnitude of current signals applied to the drive.

In one embodiment the one or more processing devices are configured to: determine a fluid viscosity indicator indicative of the viscosity of blood within the heart pump; and, calculate at least one hemodynamic parameter using the drive indicator, the bearing indicator, and the fluid viscosity indicator.

In one embodiment the one or more processing devices are configured to determine the fluid viscosity indicator at least one of: based on user input commands; and, by estimating the fluid viscosity.

In one embodiment the one or more processing devices are configured to estimate a fluid viscosity using at least one of: a plant transfer function; and, variations in the bearing indicator.

In one embodiment, the one or more processing devices are configured to estimate a presence or amount of air in the pump using at least one of: a plant transfer function; a fluid viscosity; and, variations in the bearing and/or drive indicators.

In one embodiment the parameter includes at least one of: a subject parameter; a pump operating parameter; and, a hemodynamic parameter.

In one embodiment the subject parameter includes at least one of: a periodic subject signal; a presence of a thrombus or occlusion; a location of a thrombus or occlusion; a breathing rate; a breathing depth; a subject activity level; a subject posture or subject posture change; and, at least one hemodynamic parameter, the at least one hemodynamic parameter including at least one of: a flow; a head pressure; a relative inlet pressure; one or more absolute pressures within a subject; a systemic vascular pressure; a pulmonary vascular pressure; a systemic vascular resistance; a pulmonary vascular resistance; a ratio of systemic and pulmonary vascular resistance; vascular compliance; a ventricular contractility magnitude; a ventricular contractility rate; an atrial contractility magnitude; an atrial contractility rate; a level of delivered oxygen; a cardiac event; aortic valve opening or closing; and, mitral valve opening or closing.

In one embodiment the one or more processing devices are configured to: determine at least one clinically measured pressure parameter; and, calculate remaining absolute pressure parameters, wherein the pressure parameters include: central venous pressure (CVP); left atrial pressure (LAP); pulmonary arterial pressure (PAP); and, aortic pressure (AoP).

In one embodiment the one or more processing devices are configured to at least one of: control the heart pump at least in part using the at least one parameter; display an indication of the at least one parameter; record, to non-transitory memory, an indication of the at least one parameter; and, generate an alert or notification based on at least one parameter.

In one embodiment the one or more processing devices are configured to control at least one of a rotational speed and an axial position of the impeller.

In one embodiment the one or more processing devices are configured to control a rotational speed of the impeller to at least one of: control a blood flow rate through the heart pump; vary the rotational speed to induce pulsatile flow; vary the rotational speed to induce pulsatile flow that mimics a physiological pulse; vary the rotational speed to induce pulsatile flow that matches a physiological pulse when the heart pump is used as a ventricular assist device; vary the rotational speed to induce pulsatile flow in accordance with a pulse waveform; vary the rotational speed to alter a pulse waveform of pulsatile flow; vary the rotational speed to alter a pulse waveform to at least one of: alter a pulsatile frequency; alter a pulsatile magnitude; and, change a ratio of higher speed and lower speed parts of the pulse waveform; vary the rotational speed to selectively load a subject's heart when the heart pump is used as a ventricular assist device; vary the rotational speed to selectively load a subject's heart when the heart pump is used as a ventricular assist device to thereby promote recovery of the subject's heart; increase an effective pump flow curve gradient; and, decrease an effective pump flow curve gradient.

In one embodiment the one or more processing devices are configured to: perform spectral analysis of at least one of a drive indicator and the bearing indicator; and, determine at least one subject parameter using results of the performed spectral analysis.

In one embodiment the one or more processing devices are configured to perform the spectral analysis of at least one of: an electrical current applied to the drive; and, an electrical current applied to the bearing.

In one embodiment the one or more processing devices are configured to perform filtering or signal processing to compensate for pulsatile operation of the heart pump.

In one embodiment the one or more processing devices are configured to: determine a magnitude and/or frequency of periodic signals within at least one of a drive indicator and the bearing indicator; and, determine the parameter using the magnitude and/or frequency.

In one embodiment the one or more processing devices are configured to: determine a waveform shape of signals within at least one of a drive indicator and the bearing indicator; and, determine the parameter using the waveform shape.

In one embodiment when the pump is configured to act as an assist device for a subject, the one or more processing devices are configured to: identify parts of the subject's cardiac cycle using the waveform shape; and, determine the parameter using the parts of the cardiac cycle.

In one embodiment the impeller includes first and second sets of vanes provided on a rotor body, the rotor being positioned within the cavity to define: a first cavity portion having a first inlet and a first outlet, the first set of vanes being provided within the first cavity portion so as to define a first pump that provides at least partial left ventricular function; and, a second cavity portion having a second inlet and a second outlet, the second set of vanes being provided within the second cavity portion so as to define a second pump that provides at least partial right ventricular function.

In one embodiment the axial position of the impeller determines a separation between each set of vanes and a respective cavity surface, the separation being used to control the fluid flows from the inlets to the outlets.

In one embodiment the first and second pumps have respective pump performance curves having different gradients so that a change in rotational speed of the pump causes a change in the relative flows of the first and second pumps.

In one embodiment the drive includes: a number of circumferentially spaced permanent magnets mounted in the rotor of the impeller, adjacent magnets having opposing polarities; and, at least one drive coil that in use generates a magnetic field that cooperates with the magnetic material allowing the impeller to be rotated.

In one embodiment the magnetic bearing includes: first and second annular magnetic bearing members mounted within and proximate a face of the rotor, the first magnetic bearing member being provided radially outwardly of the second magnetic bearing member; a number of circumferentially spaced substantially U-shaped bearing stators mounted in the housing proximate a second end of the cavity, each U-shaped bearing stator having first and second bearing stator legs substantially radially aligned with the first and second magnetic bearing members respectively; and, at least one bearing coil on each bearing stator that generates a magnetic field that cooperates with the magnetic bearing members to thereby at least one of: control an axial position of the impeller; and, at least partially restrain radial movement of the impeller.

In one embodiment the drive is positioned at a first end of the cavity and the magnetic bearing is positioned at a second end of the cavity.

In one embodiment the heart pump is at least one of: a ventricular assist device; and, a total artificial heart.

In one broad form, an aspect of the present invention seeks to provide a heart pump including: a housing including a cavity having first and second inlets and first and second outlets; an impeller provided within the cavity, the impeller including first and second sets of vanes; a drive configured to rotate the impeller within the cavity so that: the first set of vanes urges fluid radially from the first inlet to the first outlet; and, the second set of vanes urges fluid radially from the second inlet to the second outlet; a magnetic bearing including at least one bearing coil that controls an axial position of the impeller within the cavity; and, one or more electronic processing devices that are configured to: determine a bearing indicator relating to axial forces on the impeller; and, use the bearing indicator to calculate at least one parameter.

In one broad form, an aspect of the present invention seeks to provide a controller for a heart pump, the controller being configured to calculate at least one hemodynamic parameter, wherein the heart pump includes: a housing forming a cavity including at least one inlet and at least one outlet; an impeller provided within the cavity, the impeller including vanes for urging fluid from the inlet to the outlet upon rotation of the impeller; a drive that rotates the impeller within the cavity; a magnetic bearing including at least one bearing coil that controls an axial position of the impeller within the cavity; and, the controller including one or more electronic processing devices that are configured to: determine a bearing indicator relating to axial forces on the impeller; and, use the bearing indicator to calculate at least one parameter.

In one broad form, an aspect of the present invention seeks to provide a method of calculating at least one parameter, wherein the method includes in one or more processing devices: determining a bearing indicator associated with a magnetic bearing including at least one bearing coil that controls an axial position of an impeller within a cavity of a heart pump, wherein the bearing indicator relates to axial forces on the impeller; and, using the bearing indicator to calculate at least one parameter.

In one broad form, an aspect of the present invention seeks to provide a heart pump including: a housing forming a cavity including at least one inlet and at least one outlet; an impeller provided within the cavity, the impeller including vanes for urging fluid from the inlet to the outlet upon rotation of the impeller; a drive that rotates the impeller within the cavity; a magnetic bearing including at least one bearing coil that controls an axial position of the impeller within the cavity; and, one or more electronic processing devices that are configured to: determine a drive indicator indicative of rotation of the impeller; and, use the drive indicator to calculate the at least one parameter.

In one embodiment the drive indicator is indicative of at least one of: a current supplied to the drive; an expected rotational speed of the impeller; an actual rotational speed of the impeller; and, a magnitude of a rotational speed change.

In one embodiment the one or more processing devices are configured to: perform spectral analysis of the drive indicator; and, determine the at least one subject parameter using results of the analysis.

In one embodiment the one or more processing devices are configured to perform filtering to compensate for pulsatile operation of the heart pump.

In one embodiment the one or more processing devices are configured to: determine a magnitude and/or frequency of periodic signals within at least one of a drive indicator and the bearing indicator; and, determine the parameter using the magnitude and/or frequency.

In one embodiment the one or more processing devices are configured to: determine a waveform shape of signals within at least one of a drive indicator and the bearing indicator; and, determine the parameter using the waveform shape.

In one embodiment when the pump is configured to act as an assist device, the one or more processing devices are configured to: identify parts of the cardiac cycle using the waveform shape; and, determine the parameter using the parts of the cardiac cycle.

In one embodiment the parameter includes at least one of: a subject parameter; a pump operating parameter; and, a hemodynamic parameter.

In one embodiment the subject parameter includes at least one of: a periodic subject signal; a presence of a thrombus or occlusion; a location of a thrombus or occlusion; a breathing rate; a breathing depth; a subject activity level; a subject posture or subject posture change; and, at least one hemodynamic parameter, the at least one hemodynamic parameter including at least one of: a flow; a head pressure; a relative inlet pressure; one or more absolute pressures within a subject; a systemic vascular pressure; a pulmonary vascular pressure; a systemic vascular resistance; a pulmonary vascular resistance; a ratio of systemic and pulmonary vascular resistance; vascular compliance; a ventricular contractility magnitude; a ventricular contractility rate; an atrial contractility magnitude; an atrial contractility rate; a level of delivered oxygen; a cardiac event; aortic valve opening or closing; and, mitral valve opening or closing.

In one embodiment the subject parameter includes at least one of: a breathing rate; a breathing depth; and, at least one hemodynamic parameter, the at least one hemodynamic parameter including at least one of: vascular compliance; an atrial contractility magnitude; and, an atrial contractility rate.

In one embodiment the one or more processing devices are configured to at least one of: control the heart pump at least in part using the at least one parameter; display an indication of the at least one parameter; record an indication of the at least one parameter; and, generate an alert or notification based on the at least one parameter.

In one broad form, an aspect of the present invention seeks to provide a heart pump including: a housing including a cavity having first and second inlets and first and second outlets; an impeller provided within the cavity, the impeller including first and second sets of vanes; a drive configured to rotate the impeller within the cavity so that: the first set of vanes urges fluid radially from the first inlet to the first outlet; and, the second set of vanes urges fluid radially from the second inlet to the second outlet; a magnetic bearing including at least one bearing coil that controls an axial position of the impeller within the cavity; and, one or more electronic processing devices that are configured to: determine a drive indicator indicative of rotation of the impeller; and, use the drive indicator to calculate at least one parameter. A controller for a heart pump, wherein the heart pump includes: a housing forming a cavity including at least one inlet and at least one outlet; an impeller provided within the cavity, the impeller including vanes for urging fluid from the inlet to the outlet upon rotation of the impeller; a drive that rotates the impeller within the cavity; a magnetic bearing including at least one bearing coil that controls an axial position of the impeller within the cavity; and, wherein the controller includes one or more one or more electronic processing devices that are configured to: determine a drive indicator indicative of rotation of the impeller; and, use the drive indicator to calculate at least one parameter.

In one broad form, an aspect of the present invention seeks to provide a method performed using one or more processing devices coupled to a heart pump of a patient, the method including: determining a drive indicator associated with a drive that includes at least one drive coil that rotates an impeller within a cavity of the heart pump; and, using the drive indicator to calculate at least one parameter.

In one broad form, an aspect of the present invention seeks to provide a method of providing treatment for a subject with a heart pump, the heart pump including an impeller and a magnetic bearing, the impeller including vanes for urging fluid from an inlet of the heart pump to an outlet of heart pump upon rotation of the impeller, the magnetic bearing being configured to control an axial position of the impeller within the housing, the method including: determining an indicator relating to axial forces on the impeller or rotation of the impeller; calculating at least one parameter based on the determined indicator; and adjusting treatment for the subject based on the calculated at least one parameter.

In one embodiment the indicator includes a bearing indictor relating to axial forces on the impeller.

In one embodiment the indicator includes a drive indicator indicative of rotation of the impeller.

In one embodiment the method further includes: comparing the at least one parameter to a defined threshold; and, wherein adjustment of treatment is further based on the comparing.

In one embodiment the at least one parameter includes a subject parameter.

In one embodiment the subject parameter includes a hemodynamic parameter.

In one embodiment the at least one parameter includes an operating parameter for the heart pump.

In one embodiment adjusting the treatment includes controlling operation of the heart pump based on the calculated at least one parameter.

In one embodiment the method further includes: outputting, to a display screen, a display that includes an indication of the at least one parameter, wherein adjusting of treatment is based on the indication of the at least one parameter that is displayed.

In one embodiment the method further includes: storing, to non-transitory memory, an indication of the at least one parameter, wherein adjusting of treatment is based on the stored indication.

In one embodiment the method further includes: generating an alert or notification based on the at least one parameter, wherein adjusting of treatment is based on the alert or notification.

It will be appreciated that the broad forms of the invention and their respective features can be used in conjunction and/or independently, and reference to separate broad forms is not intended to be limiting. Furthermore, it will be appreciated that features of the method can be performed using the system or apparatus and that features of the system or apparatus can be implemented using the method.

### BRIEF DESCRIPTION OF THE DRAWINGS

Various examples and embodiments of the present invention will now be described with reference to the accompanying drawings, in which:—

FIG. 1A is a schematic perspective view of an example of a heart pump;

FIG. 1B is a schematic cutaway view of the heart pump of FIG. 1A;

FIG. 1C is a schematic perspective exploded view of the heart pump of FIG. 1A;

FIG. 1D is a schematic diagram of an example of a control system for the heart pump of FIG. 1A;

FIG. 2A is an example pump flow curve for a heart pump;

FIG. 2B is an example of pump pressure flow curves for a number of different heart pumps;

FIG. 3 is a schematic diagram of a specific example of a controller architecture;

FIG. 4 is a flow chart of an example of a process for calculating hemodynamic parameters;

FIGS. 5A and 5B are graphs showing measured atrial pressures and corresponding bearing currents respectively measured during an animal trial;

FIG. 6A is a graph of an example of comparison of measured pump flows versus flows estimated using motor drive signals only;

FIG. 6B is a graph of an example of comparison of measured pump flows versus flows estimated using motor drive signals and magnetic bearing signals;

FIG. 6C is a graph of an example of comparison of measured pump flows versus flows estimated using motor drive signals, magnetic bearing signals and fluid viscosity;

FIG. 7 is a graph of an example of a comparison of motor drive power, measured pump flow and estimated pump flow;

FIG. 8 is a flow chart of a specific example of process for calculating hemodynamic parameters;

FIG. 9 is a graph of an example of a plant transfer function used in fluid viscosity estimation;

FIG. 10 is a graph of an example of different fluid viscosities;

FIG. 11 is a graph of examples of different inlet pressures at different rotor positions and rotational speeds;

FIG. 12 is a schematic diagram illustrating patient hemodynamics;

FIGS. 13A and 13B are graphs illustrating a first example pump control scenario;

FIGS. 13C and 13D are graphs illustrating a second example pump control scenario;

FIG. 14A is a schematic perspective view of an example of a single VAD heart pump;

FIG. 14B is a schematic cutaway side view of the heart pump of FIG. 14A;

FIG. 14C is a schematic cutaway plan view of the heart pump of FIG. 14A;

FIG. 14D is a schematic perspective view of the impeller of the heart pump of FIG. 14A;

FIG. 14E is a schematic perspective view of the magnetic bearing of the heart pump of FIG. 14A;

FIG. 14F is a schematic cutaway perspective view of the impeller of FIG. 14A.

FIG. 15 is a flow chart of an example of the process for calculating periodic subject parameters;

FIG. 16A is a graph of an example of magnetic bearing current signals captured for a subject during a monitoring period;

FIG. 16B is a graph of an example of impeller position captured for the subject during the monitoring period of FIG. 16A;

FIG. 16C is a graph of an example of vascular resistance for the subject during the monitoring period of FIG. 16A;

FIG. 16D is a graph of an example of pump inlet pressure differences captured for the subject during the monitoring period of FIG. 16A; and,

FIG. 17 is an example of a waterfall plot showing periodic subject parameters derived for the monitoring period of FIG. 16; and,

FIG. 18 is an example of a plot showing an increase in impeller vibration.

### DETAILED DESCRIPTION OF THE PREFERRED EMBODIMENTS

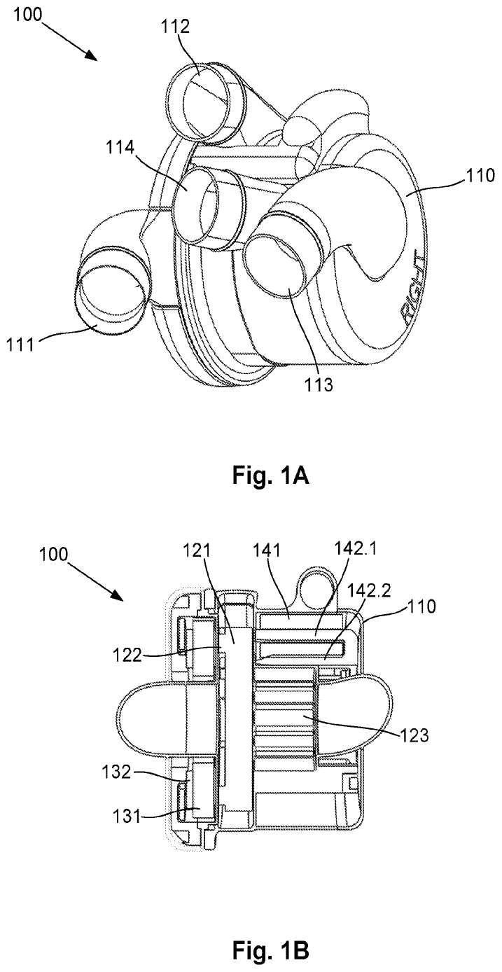

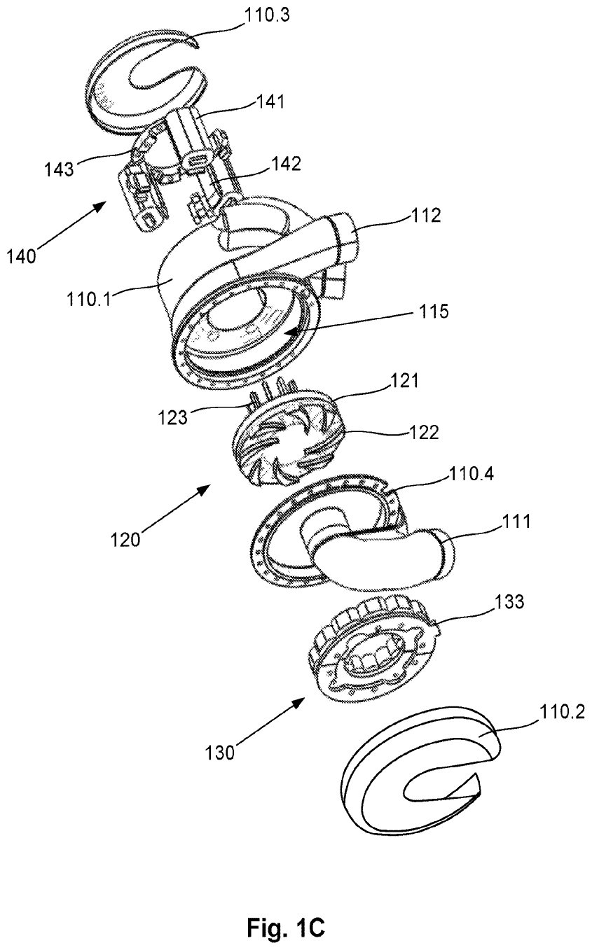

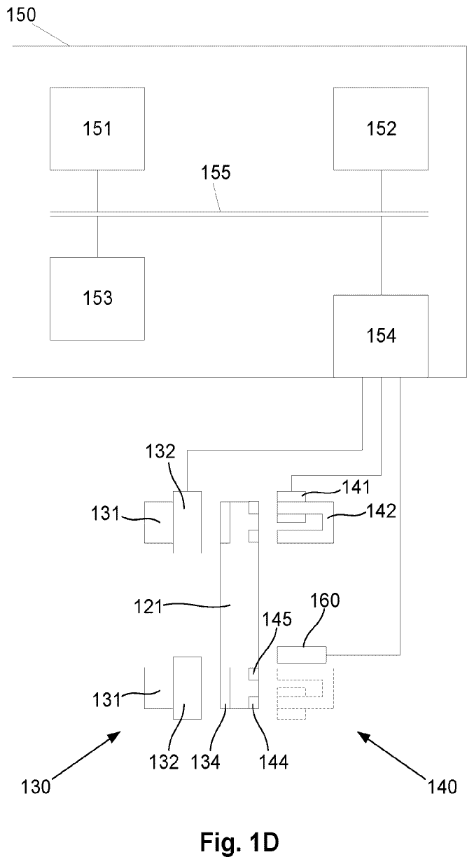

An example of a heart pump will now be described with reference to FIGS. 1A to 1D.

In this example the heart pump is a biventricular device which can operate either as a ventricular assist device to assist function of left and right ventricles of a subject's heart, or alternatively as a total artificial heart. It will be appreciated however that whilst reference is made to a biventricular device this is not essential, and alternatively the control processes described herein could equally be applied to single ventricular assist devices or any form of blood pump.

In this example, the heart pump 100 includes a housing 110 defining a cavity 115. The housing can be of any suitable form but typically includes a main body 110.1, left and right end caps 110.2, 110.3 which connect to the main body 110.1, as well as an end plate 110.4 positioned between the main body 110.1 and left end cap 110.2. The housing can be made of any suitable biocompatible material, and can be made of titanium, a polymer or the like.

The housing 110 includes two inlets 111, 113, for connection to the pulmonary vein and vena cava, or left and right ventricles, and two outlets 112, 114 for connection to the aorta and pulmonary artery, respectively. Whilst two inlets and outlets are shown, it will be appreciated that this is in the context of a biventricular device, and that a single inlet and outlet can be used for a single ventricular device.

The heart pump 100 includes an impeller 120 provided within a cavity 115. The impeller 120 includes a rotor 121 having vanes mounted thereon for urging fluid from the inlet to the outlet upon rotation of the impeller 120. In this example, as the heart pump 100 is a biventricular device, the impeller includes two sets of vanes 122, 123 each of which is used for urging fluid from a respective inlet 111, 113 to a respective outlet 112, 114. In this example, the rotor 121 is positioned within the cavity 115 to effectively divide the cavity into first and second cavity portions, each having a respective inlet and outlet, thereby allowing each to function as a respective pump.

Thus, in the current example, the vanes 122 are used to urge fluid from the inlet 111 to the outlet 112, with this being provided on the left-hand side of the pump in the orientation shown in FIG. 1B, and operating to provide left-ventricular function, whilst the vanes 123 urge fluid from the inlet 113 to the outlet 114 and act to provide right-ventricular function. In this context the first and second cavity portions are generally referred to as left and right cavities, respectively. It will be appreciated that in this regard, the terms left and right refer to the intended ventricular function of the cavities as opposed to the particular orientation of the pump in FIGS. 1B, which is used for illustrative purposes only.

The heart pump 100 further includes a drive 130 that rotates the impeller 120 within the cavity 115. The drive 130 can be of any appropriate form but typically includes a number of coils 131 wound on a stator 132, supported by a mounting 133, allowing the drive 130 to be coupled to the housing 110. The drive cooperates with magnetic material 134 mounted in the rotor 121 with this typically being in the form of a number of circumferentially spaced permanent magnets mounted in the rotor 121 proximate an outer circumferential edge of the rotor and proximate a face of the rotor facing the drive coils 131. In one specific example, the coils 131 and stators 132 are wedge shaped and circumferentially spaced around the mounting 133, so as to provide twelve electromagnets radially aligned with circumferentially spaced drive magnets 134 in the rotor 121, to thereby maximise a degree of magnetic coupling between the magnets in the rotor 121 and the drive 130. The drive magnets 134 are typically arcuate shaped rare earth magnets, circumferentially spaced proximate an outer circumferential edge of the rotor 121, and mounted on a soft iron rotor drive yoke.

The heart pump 100 can further include a magnetic bearing 140 including at least one bearing coil 141 which cooperates with bearing magnetic material mounted in the rotor 121 allowing to thereby control an axial position of the impeller 120 within the cavity 115. In one particular example, the magnetic bearing includes three bearing coils 141, each of which is mounted on a first leg 142.1 of respective U-shaped stators 142, with a second leg 142.2 being positioned radially inwardly of the first leg 142.1. The stators 142 are mounted to a support 143 and circumferentially spaced 120° apart around the housing so that the first and second legs 142.1, 142.2 align with respective magnetic material, such as bearing magnets 144, 145, optionally mounted on a common yoke (not shown) allowing an axial position of the impeller 120 to be controlled.

The bearing magnetic material typically includes first and second annular magnetic bearing members mounted within and proximate a face of the rotor facing the bearing coils 141, the first magnetic bearing member being provided radially outwardly of the second magnetic bearing member. In one particular example, the first bearing magnet material 144 includes an annular soft iron material that can be integrally formed with the annular yoke, or an annular permanent magnet 144 mounted on the yoke, and mounted in the rotor, proximate an outer circumferential edge of the rotor 121. The second bearing magnetic material is an annular permanent bearing magnet 145 mounted radially inwardly of the first bearing member 144, so that the first and second bearing members 144, 145 align with respective legs 142.1, 142.2 of the stators 142. It will be appreciated the annular members could include a plurality of individual elements, such as individual circumferentially spaced magnets or ferromagnetic elements. Additionally, the use of a magnetic bearing may not be required and can be replaced by a static physical bearing, or the like.

The drive 130 and magnetic bearing 140 are mounted at opposing ends of the housing 110 so that the drive and bearing 130, 140 are provided proximate opposing surfaces of the rotor 121 as shown for example in FIG. 1D. In the current example the drive 130 is mounted adjacent the left pump, whilst the bearing 140 is mounted adjacent the right pump, although the opposite configuration is contemplated. The depicted arrangement has a number of benefits.

Firstly, the inherent attractive magnetic forces between the drive and rotor and the bearing and rotor can be configured to substantially balance when the rotor is provided at a balance point at a normal operating speed, which may for example by approximately at a center of the cavity under conditions of normal hemodynamic conditions.

For example, this arrangement can be configured so that the magnetic forces inherent between the drive 130 and impeller 120, and between the magnetic bearing 140 and impeller 120 are matched at an impeller balance position within the cavity, which corresponds to a desired position of the impeller under conditions of normal flow. This minimises the bearing current required to maintain the position of the impeller 120 within the cavity, hence reducing the amount power required to operate, and in particular drive and axially position the impeller.

Additionally, as well as having the magnetic forces balance, the forces generated by the drive and bearing can also be configured to provide a desired degree of axial and radial stiffness. In this regard, the stiffness is a measure of the deflection of the impeller 120 from a balance position in response to an external force. In one example, it is desirable to maximise the radial stiffness so as to maintain the impeller radially centralised within the cavity and to stop the impeller touching the inner circumferential wall of the cavity. Conversely, as the axial position of the impeller 120 can be used for flow control, and in particular to allow for passive and/or active response to changes in hemodynamic parameters, a low degree of axial stiffness is preferred. Accordingly, the passive magnetic forces can be configured to assist in meeting these requirements, as will be described in more detail below.

A further benefit of the above described arrangement, in the context of BiVAD applications, is that it allows the greater size of the magnetic bearing to be accommodated by the smaller size of the right pump cavity. In particular, this allows a gap between a bearing stator and bearing magnets to be minimized, as no vanes are located in this gap (as opposed to the left side where vanes are located in the magnetic airgap between the drive and the rotor), as will be described in more detail below. However, it will be appreciated that this limits an outer diameter of the right pump and thus achievable pressure generation at a given rotational speed, although for right pumps this is generally not an issue given their lower flow requirements than the left pump. As shown in FIG. 1D, the apparatus further includes a controller 150 which, in use, is coupled to a sensor 160 and the drive and bearing coils 131, 141. The sensor 160 senses an axial position of the impeller 120 within the cavity 115 and can be of appropriate form such as a reluctance or eddy current sensor, which detect magnetic fields within the rotor 121 to thereby determine a separation between the rotor and the sensor 160, as will be appreciated by persons skilled in the art.

Typically three sensors would be provided circumferentially spaced around the rotor. In the case of eddy current sensors 160, each sensor would typically include a coil mounted in a housing, circumferentially spaced and aligned with the inner second leg 142.2 of the magnetic bearing stators 142. The coil is aligned with a rotor shell/target mounted radially inwardly of a first bearing magnet 144, so as to generate a field therein, with variations in the field being detected to determine the separation of the sensor 160 and the shell/target, and hence the rotor 121. However, it will be appreciated that other suitable sensors can be used, such as reluctance sensors or the like, in which case the first permanent magnet 144 might be replaced with ferromagnetic material, depending on the sensor/bearing requirements.

In use the controller 150 is adapted to monitor signals from the position sensor 160 and then control the current supplied to the drive coils 131 to control rotation of the impeller and to the bearing coils 141 to control the axial position of the impeller 120. Thus, the impeller 120 is acted upon by the fluid pressures in the housing 110, which create a net hydraulic force on the impeller 120. Forces acting on the impeller 120 are compensated for by the magnetic bearing, with the controller 150 operating to control the amount of current supplied to the electromagnets in the bearing to thereby maintain the position of the impeller 120. As such, the current used by the magnetic bearing system has a direct correlation to the forces and pressures acting on the impeller 120. In this manner, changes to the inlet and outlet pressures can be detected through the magnetic bearing signals in real-time.

The controller 150 can be of any suitable form but typically includes an electronic processing device 151, an optional memory 152, and an interface 154 for connecting to the heart pump, each of which are interconnected by a bus 155, or other similar arrangement. The electronic processing device can be any form of electronic processing device capable of interpreting signals and causing the drive and bearing to be controlled, such as a microprocessor, microchip processor, logic gate configuration, firmware optionally associated with implementing logic such as an FPGA (Field Programmable Gate Array), or any other electronic device, system or arrangement. The system can use multiple processing devices, with the indicated processing being performed by any or all of the one or more of the processing devices. For ease of illustration, the examples herein are discussed in connection with an “electronic processing device” (or a “processing device”). However, it will be appreciated that reference to a processing device or similar should be understood to encompass both a single processing device and multiple processing devices performing the described actions, operations, or the like, In the case of multiple processing devices, processing carried out by each instance of a processing device may be distributed as appropriate. Indeed, unless otherwise specified, reference to an electronic processing device (including one or more electronic processing devices) being configured to perform operations (and/or performing such operations) should be understood such that each performed action, operation, or the like, may be performed by any of the different electronic processing devices, the same electronic processing device, or any combination thereof. Furthermore, whilst reference is made to a controller in connection with some examples, it will be appreciated that one or more processing devices used for calculating parameters (e.g., hemodynamic parameters) could be separate to the controller. For example, the system could include a controller with a first processing device and a second remote processing device, with calculation of parameters being performed by either processing device, or split between the processing devices as needed, or provided by a processing device that is separate from the controller. In some embodiments, the one processing device may be provided in the body of the subject (e.g., with an implanted heart pump) while a second processing device may be provided externally (e.g., on a mobile device, a bedside device, in a cloud computing environment, etc.). Accordingly, the processing techniques herein may be distributed among various processing devices.

An optional external interface 153 may be provided allowing for interaction with the controller 150. In the event that the controller is positioned outside the body this could include an I/O device 153 such as a touch screen, display, or the like, whereas if positioned inside the body this would typically be in the form of a wireless communications module allowing communication with an external control device, a processing system, such as a computer system, a client device, such as a smart phone, a patient monitoring system or similar.

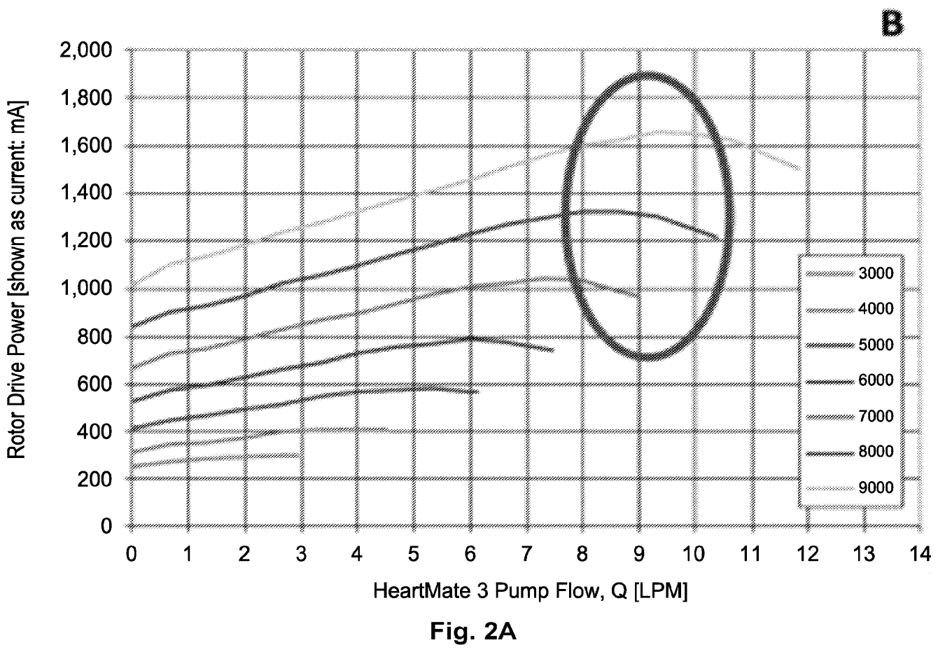

Typically, the mean flow of a left ventricular assist device (LVAD) is estimated based on a known relationship between the motor power and the pump flow for a given pump speed. An example of this is shown in FIG. 2A. The pressure head can be estimated using a similar relationship, as shown in FIG. 2B. However, the viscosity of the working fluid can change the relationship between the motor power and output flow, so hematocrit needs to be considered in LVAD flow estimation.

Nevertheless, estimation of flow is not necessarily accurate taking into account motor power and/or speed and haematocrit. For example, as shown in FIG. 2A, the pump flow curve undergoes an inflexion, meaning two different pump flows can be obtained for a given motor power, meaning there is ambiguity when calculating flow based on the motor speed. Additionally, such approaches will not work on dual sided impeller designs similar to those described above.

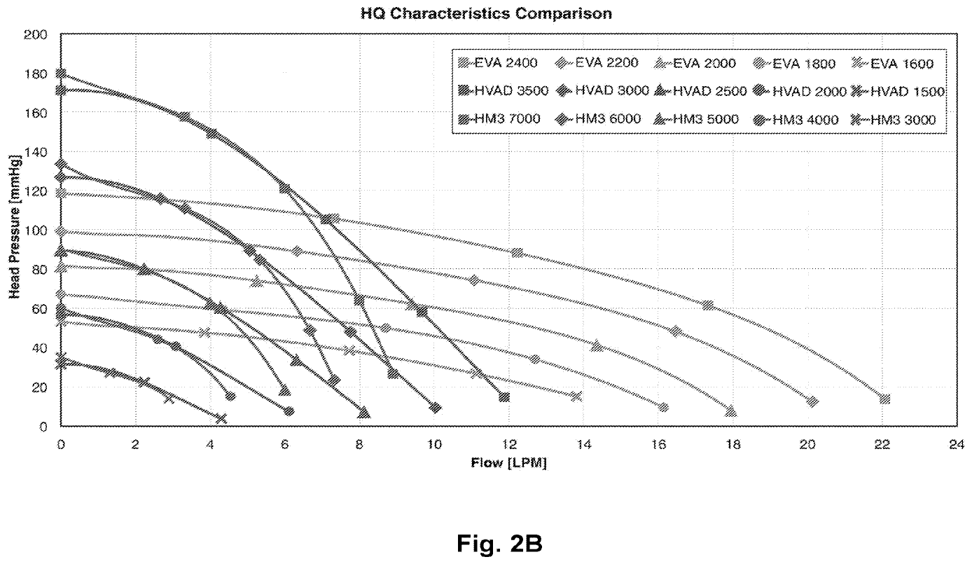

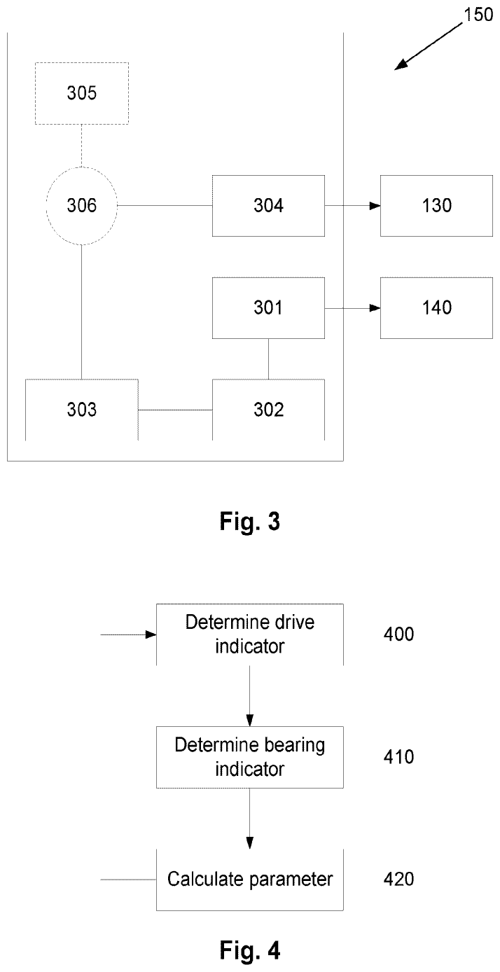

A specific example of a controller 150 for controlling the drive 130 and bearing 140 will now be described with reference to FIG. 3.

In this example, the controller implements a bearing controller 301 which operates to maintain the impeller 120 at a target axial position within the cavity. The target axial position can be determined in accordance with zero power operations described for example in U.S. Pat. No. 8,636,638 or WO2017120453, although this is not essential and any suitable bearing control strategy can be used.

In any event, the controller further includes a current sensor 302 which monitors the bearing power indicator providing any indication of this to a processor 303. The processor 303 calculates the change in required impeller rotational speed, generating a drive control, which is provided to a drive controller 304, which controls the rotational speed of the impeller accordingly. Additionally, an optional drive signal modulator 305 can be provided to generate a modulating signal for modulating the rotational speed of the impeller with this being combined with the signal output from the processor 303 by suitable logic, an amplifier or the like, at 306.

An example of an alternative approach for calculating parameters, and in one particular example, subject parameters such as hemodynamic parameters, will now be described with reference to FIG. 4.

For the purpose of illustration, it is also assumed that the process is performed at least in part using one or more electronic processing devices, optionally forming part of the controller 150.

In this example, at step 400 the processing device operates to determine a drive indicator drive indicator at least partially indicative of rotation of the impeller, such as the power used by the drive 130 and/or the rotational speed of the impeller 120.

At step 410, the processing device determines a bearing indicator relating to axial forces on the impeller. The bearing indicator is typically at least partially indicative of operation of the magnetic bearing and can be indicative of an axial position of the impeller but more typically is indicative of a bearing power used by the magnetic bearing. In particular, the bearing power indicator is typically indicative of, or derived from, the bearing current drawn by the magnetic bearing. However, it will be appreciated that this is not essential, and any suitable bearing indicator can be used.

At step 420 the controller uses one or both of the drive indicator and the bearing indicator to determine one or more parameters. The parameters can include subject parameters, such as hemodynamic parameters, including a head pressure, a relative inlet pressure, absolute pressures within a subject, a systemic vascular pressure or a pulmonary vascular pressure, a measure of delivered oxygen, or the like. Alternatively, the subject parameters can be indicative of periodic events within the subject, such as a breathing rate, breathing depth, vascular compliance, atrial contraction, ventricular contractility (in the case of the VAD being an assist device), or the like. Furthermore, the parameters could be indicative of other subject parameters, such as the presence of a thrombus, partial occlusion of the inflow or outflow conduits, for example caused by a kink in the graft or misalignment of the inflow cuff, or the like, as well as pump parameters or pump operational performance parameters.

In general, when determining hemodynamic parameters, the approach uses both of the drive and bearing indicators. However, when determining periodic parameters, only one of the drive and/or bearing indicators may be required, in which case it will be appreciated that the other parameter need not be determined in either step 400 or step 410.

The manner in which the parameters are calculated will vary depending on the preferred implementation and the nature of the parameter being calculated. For example, this can involve analysing the bearing and/or drive indicator using a variety of signal processing techniques, including but not limited to performing spectral analysis, such as Fourier transforms, using linear equations (e.g. polynomials), or non-linear functions, and filtering, such as Kalman filtering, performing a convolution, cross correlation, or auto correlation, regression analysis, or the like. These techniques can be used to identify features within the bearing or drive indicators, such as periodic signals that are indicative of physiological parameters, or the like. Specific example approaches for different parameters will be described in more detail below.

Following this, the controller can optionally control the pump, for example controlling the drive 130 to change a rotational speed of the impeller, or controlling the bearing to adjust an axial position of the impeller, based on the one or more hemodynamic parameters and/or other subject parameters. Additionally and/or alternatively, the controller can store and/or output the one or more subject or hemodynamic parameters, allowing these to be used in ongoing patient monitoring, clinical diagnosis, clinical intervention, alerting or notification, or the like. For example, this could be used to alert a clinician of an issue, so the clinician can perform an assessment or intervention, or could be used to warn the patient for example, indicating to the patient to reduce activity levels or similar.

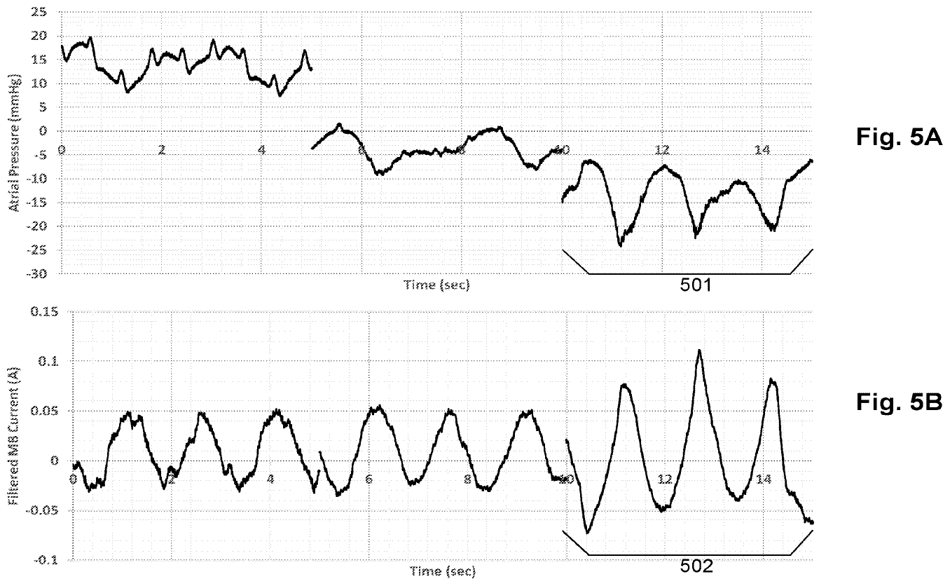

Accordingly, in one example, the above described approach uses a bearing indicator to improve estimation of the hemodynamic parameters. In this regard, the axial forces on an impeller vary depending on hemodynamic parameters, and an example of this is shown in FIGS. 5A and 5B, which show variations in arterial pressure and bearing current for a calf during an animal trial.

In particular, FIG. 5A shows a range of different atrial pressures with a low pressure event being represented by the portion of the wave form shown at 501. Notably, the waveforms include peaks and troughs corresponding to perturbations in the speed of the impeller. The corresponding magnetic bearing current for each atrial pressure is shown in FIG. 5B, which as shown at 502 demonstrates significantly greater peaks and troughs for low pressure conditions that then higher pressure conditions, exemplifying that monitoring the operation of the magnetic bearing allows for detection pressure variations, which used in conjunction with information derived from the drive indicator, allows hemodynamic parameters to be determined.

Accordingly, it will be appreciated that taking into account both the drive and bearing indicators can lead to an improvement in the ability to accurately assess hemodynamic parameters in VADs, or other similar devices, whilst allowing hemodynamic parameters to be calculated in BiVADs, which is not currently achievable.

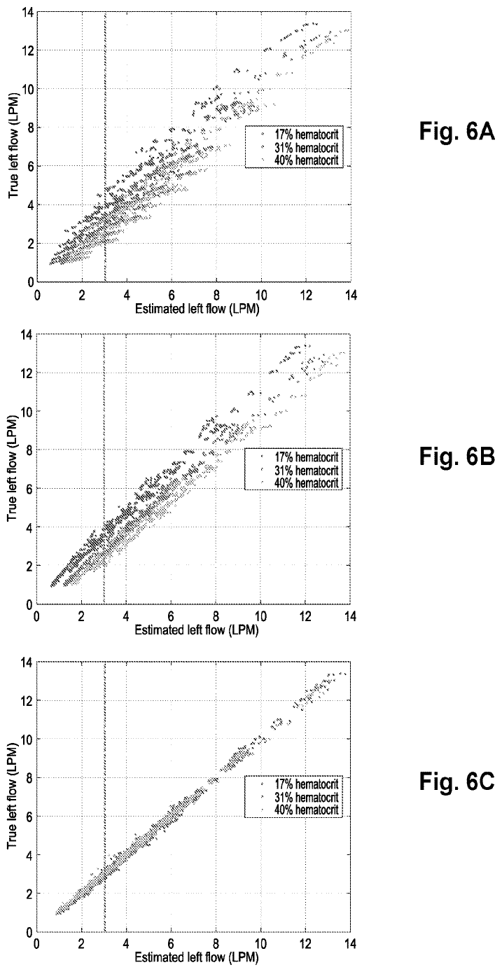

An example of this is shown in FIGS. 6A to 6C, which illustrate a comparison of measured and predicted left flow for a heart pump. In the example of FIG. 6A flow is estimated using a drive indicator only, which proves to be of only very limited accuracy. Addition of the bearing indicator results in significant improvement, as shown in FIG. 6B, with the only inaccuracies arising due to changing hematocrit, which in turn impacts on fluid viscosity. Taking this into account can lead to further improvements, as shown in FIG. 6C, in which the predicted flow is an extremely accurate estimate of actual flow.

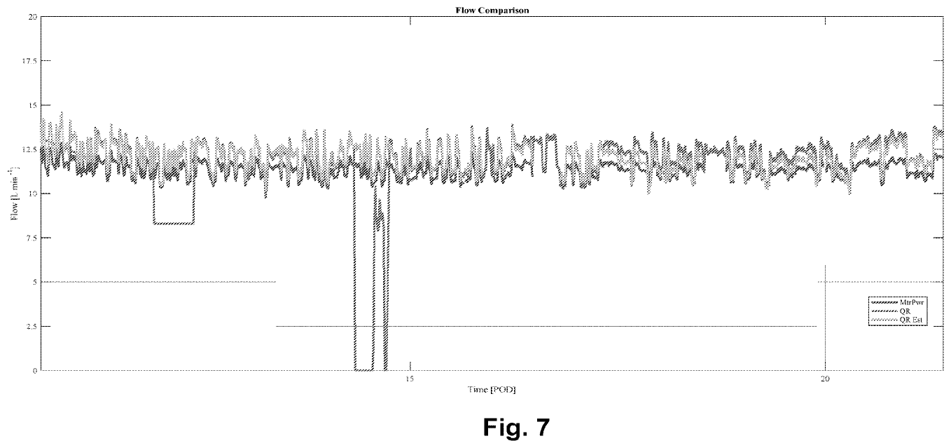

A further flow comparison showing accurate tracking of actual flow is shown in FIG. 7.

In another example, monitoring of the bearing and/or drive indicators can be used to determine other subject parameters, particularly periodic parameters, such as a breathing rate, or the like. Determination of subject parameters of this form can be used to monitor for issues, such as excessive or repressed breathing. This in turn can be used to control operation of the heart pump, for example increasing or decreasing the pump flow or speed to adjust an amount of oxygen delivered to the body, and/or to generate a notification, for example to alert a medical practitioner to an adverse event. In respect of altering the pump flow, it will be appreciated that the pump can be configured to provide pulsatile flow by modulating the pump speed, in which case the pump flow could be altered by adjusting the speed, or adjusting the pulsatile flow, for example, by increasing a pulse rate (frequency), pulse shape or profile and/or pulse magnitude, to thereby increase the mean flow rate.

A number of further features will now be described.

The bearing indicator is typically indicative of an electrical current used by the magnetic bearing 140 but could additionally or alternatively be indicative of another aspect of operation of the bearing 140, such as a tilt of the impeller 120 relative to the bearing 140 and/or an axial and/or radial position of the impeller 120 within the cavity, depending upon the control strategy used by the heart pump. As previously mentioned, the bearing indicator can be simply indicative of a change in indicator, and hence either a change in tilt, electrical current or axial or radial position of the impeller, but could also be indicative of a derivative or rate of change, an integral, or a magnitude in difference between maximum and minimum values over a pulse period. These approaches can provide greater sensitivity, for example to the onset of low pressure events. The bearing indicator could also be based on multiple degrees of freedom, such as the tilt and the impeller axial position.

The bearing indicator can be determined based on signals from a position sensor configured to detect an axial and/or radial position of the impeller and/or based on a current supplied to the at least one bearing coil, depending on the configuration of the heart pump and/or the preferred implementation.

In one example, the drive indicator can be indicative of any aspect of drive operation and could include a current supplied to the drive 130, an expected rotational speed of the impeller 120, an actual rotational speed of the impeller 120 and a magnitude of a rotational speed change. In one particular example, the controller is configured to determine the drive indicator based on a magnitude of current signals applied to the drive 130.

In one example, the controller is configured to determine a fluid viscosity indicator indicative of the viscosity of blood within the heart pump and use the drive indicator, the bearing indicator and the fluid viscosity indicator to calculate the at least one hemodynamic parameter. Further including the fluid viscosity in the calculation of hemodynamic parameters can lead to a further improvement in the accuracy of the calculation.

The fluid viscosity indicator can be determined based on user input commands, for example by having a clinician provide an indication of hematocrit (HCT), or can be determined by estimating the fluid viscosity. This can be achieved using known techniques, such as by using a plant transfer function and/or variations in the bearing indicator, as will be described in more detail below. Similarly, the approach can be used to estimate a presence or amount of air, with this typically being performed using the plant transfer function, the fluid viscosity and/or variations in the bearing and/or drive indicators.

The above approach can be used to calculate a variety of different hemodynamic parameters including any one or more of a flow, a head pressure, a relative inlet pressure, one or more absolute pressures within a subject, a systemic vascular pressure, a pulmonary vascular pressure, a systemic vascular resistance, a pulmonary vascular resistance, a ratio of systemic and pulmonary vascular resistance, vascular compliance, a ventricular contractility magnitude, a ventricular contractility rate, an atrial contractility magnitude, an atrial contractility rate, a level of delivered oxygen, a cardiac event, such as aortic valve opening or closing or mitral valve opening or closing.

In general, only relative pressures can be determined without further external input. However, if the controller can determine at least one clinically measured pressure parameter, for example via signals from a sensor, or based on manual input, then it is possible to calculate remaining absolute pressure parameters. Thus, by providing one of the following parameters, this allows all of the following absolute pressure parameters to be determined including central venous pressure (CVP), left atrial pressure (LAP), pulmonary arterial pressure (PAP) and aortic pressure (AoP). For example, a mean arterial pressure (MAP) can be estimated from pressure cuff, whilst CVP can also be measured non-invasively, whilst a Cardiomems implantable pressure sensor can be used to measure PAP. It will also be appreciated that in some circumstances, measures can be inferred, for example increasing pump flow to identify commencement of suction can be used to identify an absolute pressure, which can be performed as part of a washout pulse.

When pressures are known, for example if two pressures are known from measurement, such as CVP and AoP, then HCT can be estimated. Thus, in this instance, instead of estimating system pressures from motor power, motor speed, position and HCT, you can solve for HCT and use pressure difference as an input.

In one particular example, the controller calculates left and right pump pressures and an inlet pressure difference based on polynomial equations having coefficients established by measuring pump operating parameters under various conditions. These values are then used to calculate other ones of the hemodynamic parameters, based on known relationships between the parameters, as will be described in more detail below. However, it will be appreciated that other techniques could be used, including but not limited to linear equations (e.g. polynomials), non-linear functions, and filtering, such as Kalman filtering.

In addition to determining hemodynamic parameters, the arrangement can also determine a periodic subject signal, such as a breathing rate or breathing depth, as well as a subject activity level. The activity level can be indicative of a level of activity being undertaken, and could include information such as whether the subject is resting, sleeping, moving, walking, running or the like. In one example, this information can be derived from other parameters, such as the breathing rate or depth, the pump flow rate, ventricular/atrial contraction, or the like. Additionally, and/or alternatively, activity detection could be achieved by monitoring the bearing and/or drive indicators and identify patterns in the indicators, such as periodic events, which can in turn be used to identify activities, such as walking, running or the like. Similarly, a subject posture or subject posture change can be determined, for example using changes in bearing indicator to indicate a shift in physical posture, and/or using the bearing and/or drive indicators to determine a directional of a gravitational force on the impeller, which can in turn be indicative of a subject orientation.

Having determined the one or more parameters, then the determined one or more parameters may then be used in various ways. In some examples, the calculated one or more parameters may be used by, for example, a clinician to adjust therapy/treatment that is provided to a subject. Examples of adjusting therapy or treatment may include modifying operation of the pump (e.g., to pump less or more) and/or providing a recommendation/adjustment to an aspect of a subject's lifestyle (e.g., to lower their level of exertion). As discussed herein, such adjustments may be delivered to/by the patient, clinician, or others and may be carried out manually or automatically (e.g., via adjusting the pump) in some examples.

In some examples, the processing device of the controller 150 can control the pump based on the parameter. For example, this could include controlling an impeller axial position and/or a rotational speed of the impeller. This can assist in allowing the pump to be automatically controlled in response to physiological changes in the subject. This can include for example performing flow control or balancing, to maintain hemodynamic parameters within target ranges, adjusting the pump to account for subject activity, for example increasing or decreasing pump flow as the subject becomes more or less active.

For example, the processing device can control a blood flow rate through the heart pump, adjusting this to meet physiological requirements of the subject as needed, for example increasing the flow rate when the subject is exercising or decreasing the flow rate when the subject is resting. The physiological requirements can be determined from subject parameters, such as hemodynamic parameters, information regarding ventricular or atrial contractility, subject activity levels, or other parameters.

The processing device can vary the rotational speed to induce pulsatile flow, for example to produce a washout pulse to reduce stagnation within the pump and/or to vary the rotational speed to induce pulsatile flow that mimics a physiological pulse.

When the pump is used as a ventricular assist device this can also be used to generate pulsatile flow that matches a physiological pulse of the subject and/or to selectively load the subject's heart. For example, the processing device can determine the subject's physiological pulse based on parameters such as valve opening/closing or ventricular or atrial contractility, allowing the pump to match the cardiac cycle, to ensure optimum assistance is provided. Additionally, selectively loading a subject's heart can help promote recovery of the subject's heart. For example, when operating the pump as a ventricular assist device, completely and continuously unloading the left ventricle means that the left ventricle muscle has minimal work to do and therefore wouldn't regenerate or recover. However, by periodically unloading the left ventricle by reducing pump speed or configuring the pump speed to create a valve pressure to prevent backflow rather than pump and support forward flow, then the left ventricle is required to perform work, which can promote left ventricle recovery. By way of an example, the pump can be operated to monitor left ventricle contractility, progressively increasing the magnitude or duration of load on the ventricle (by reducing pump flow) over time and/or when improvements in left ventricular contractility are detected. It will also be appreciated that this could be performed in conjunction with other interventions, such as stem cell therapy, or the like, as described in “Stem Cell and Left Ventricular Assist Device Combination Therapy-A Novel Approach Aiming to Achieve Myocardial Recovery in Patients With Advanced Chronic Heart Failure” by Gregor Poglajen, Igor D. Gregoric, Rajko Radovancevic and Bojan Vrtovec, in Circulation: Heart Failure. 2019; 12:e005454, or “Can stem cell therapy increase the rate of myocardial recovery in left ventricular assist device-supported advanced heart failure patients?—current data and future perspectives” by Gregor Poglajen, and Bojan Vrtovec, Ann Transl Med. 2019 November; 7(22): 613.

The processing device can vary the rotational speed to induce pulsatile flow in accordance with a pulse waveform. The pulse waveform will typically include peaks corresponding to higher operating speeds and troughs corresponding to lower operating speed, with these corresponding to periods of systolic and diastolic flow, respectively. Depending on measured parameters, the rotational speed can be controlled to alter a pulse waveform of pulsatile flow, for example to alter a pulsatile frequency, alter a pulsatile magnitude and/or change a ratio of higher speed and lower speed parts of the pulse waveform.

Additionally, and/or alternatively, the parameter that is determined may be displayed (e.g., on a display screen to a clinician) and/or recorded (e.g., for further follow-up). The recorded and/or displayed information regarding the determined parameter may then be used by a clinician or other medical practitioner. As an illustrative example, a clinician or other medical practitioner can review the determined parameters, such as hemodynamic parameters, and use these to: 1) monitor the subject. For example, to check that the subject is responding correctly to the heart pump, and the heart pump is working correctly, and the like. As another illustrative example, a clinician or other medical practitioner can use any or all of the determined parameter(s) to make a clinical diagnosis for the subject or a therapy determination for the subject. This allows, for example, interventions to be performed as required. For example, by adjusting operation of the pump (e.g., based on clinician provided settings), or otherwise treating the subject, for example by providing medications, or adjusting patient lifestyle or other behaviours.

Additionally, and/or alternatively, the parameter that is determined may be further processed and used to, for example, generate one or more alerts or notifications. Further processing of the parameter may be performed by comparing, using a processing device, the one or more parameters to reference ranges, thresholds, and/or the like. Ranges may represent expected values for the parameter in question. Thresholds may represent upper and lower bounds for such parameters. Generating an alert or notification can then be performed depending on results of the comparison.

The alert or notifications could be provided to the subject and/or a clinician, depending on the nature of the issue. For example, if it is determined the subject is over exerting themselves (e.g., based on determination of one or more parameters and comparison to a range or threshold), a notification could be presented to the subject informing them to reduce their physical activity. Alternatively, if it is determined the patient is responding adversely to current pump operation a clinician could be alerted. The nature of the alert or notification could vary depending on the preferred implementation and the nature of any issue. For example, visual and/or audible indications could be provided via an input/output device of a controller, a message could be sent to a client device, such as a phone, computer system, or the like.

In one example, the controller 150 is configured to modify (e.g., based on determination of one or more parameters) the speed as a function of flow or pressure so that the rotational speed of the impeller is controlled to increase or decrease an effective pump flow curve gradient. Thus, this can be used to increase or decrease the change in head pressure that is achieved for a change in flow rate, in turn provide a greater degree of control over the flow and pressures within the subject.

When the heart pump is configured to determine periodic parameters, such as a breathing rate, the processing device typically performs spectral analysis, such as a Fourier analysis of the drive indicator and/or bearing indicator and determines the at least one subject parameter using results of the analysis. Typically this is performed using the electrical current applied to the drive and/or bearing, with optional filtering being used to compensate for pulsatile operation of the heart pump, as will be described in more detail below. Additionally and/or alternatively, other processing techniques that could be used include performing a convolution, cross correlation, or auto correlation, regression analysis, fitting to linear or non-linear functions, performing filtering, such as Kalman filtering, or the like.

In one example, the processing device is configured to determine a magnitude and/or frequency of periodic signals within the drive indicator and/or bearing indicator, for example looking for periodic signals within the drive and/or bearing current. The magnitude and/or frequency of these signals can then be used to determine parameters. Alternatively, the processing device can determine a waveform shape of signals within at least one of a drive indicator and the bearing indicator and then determine the parameter using the waveform shape. For example, when the pump is configured to act as an assist device for a subject, this could involve detecting signal corresponding to the subject's heartbeat, and then identifying parts of the subject's cardiac cycle using the waveform shape so that these could be used to determine the parameter. For example knowledge of the timing of diastole and systole can help identify events such as valve opening and/or closing, and/or to modify or set the speed of the pump to provide improved interaction between the native cardiac function and the heart pump. For example, as described above, this can be used to control the pump to match the cardiac cycle, to ensure optimum assistance is provided, as well as selectively loading or unloading the heart in order to promote recovery of the heart.

In one example, the pump includes a single sided impeller configured to act as a VAD, as will be described in more detail below. In another example, the impeller is a dual sided impeller similar to the example described above. In this example, the impeller includes first and second sets of vanes provided on a rotor body, with the rotor being positioned within the cavity to define a first cavity portion having a first inlet and a first outlet, the first set of vanes being provided within the first cavity portion so as to define a first pump that provides at least partial left ventricular function and a second cavity portion having a second inlet and a second outlet, the second set of vanes being provided within the second cavity portion so as to define a second pump that provides at least partial right ventricular function.

In this dual sided impeller example, an axial position of the impeller within the cavity determines a separation between each set of vanes and a respective cavity surface, the separation being used to control the fluid flows from the inlets to the outlets.

Typically the vanes of the impeller have different configurations, such as different sizes, shapes and number of vanes, so that the first and second pumps have respective pump performance curves having different gradients so that a change in rotational speed of the pump causes a change in the relative flows of the first and second pumps.

In one example, the drive includes a number of circumferentially spaced permanent magnets mounted in the rotor of the impeller, adjacent magnets having opposing polarities and at least one drive coil that in use generates a magnetic field that cooperates with the magnetic material allowing the impeller to be rotated.

Similarly, the magnetic bearing typically includes first and second annular magnetic bearing members mounted within and proximate a face of the rotor, the first magnetic bearing member being provided radially outwardly of the second magnetic bearing member, a number of circumferentially spaced substantially U-shaped bearing stators mounted in the housing proximate a second end of the cavity, each U-shaped bearing stator having first and second bearing stator legs substantially radially aligned with the first and second magnetic bearing members respectively and at least one bearing coil on each bearing stator that generates a magnetic field that cooperates with the magnetic bearing members to thereby control an axial position of the impeller; and/or at least partially restrain radial movement of the impeller.

With such an arrangement, the drive is typically positioned at a first end of the cavity and the magnetic bearing is positioned at a second end of the cavity.

When performing monitoring described above, for example to determine hemodynamic and/or periodic subject parameters, the subject can be placed into a known position, such as standing or supine, to improve the performance of the estimators, which ensures that the effect of gravity on rotor position or current is in a known direction and consistent between measurements.

Known perturbations, such as a periodic pulse, washout events and triggered events which are instigated by the controller can also be used to determine additional information from the system. For example, a washout pulse is often used to flush the pump and avoid stagnation in the pump. During this process, the pump is typically operated at a constant rotational speed (either significantly higher or lower than the normal mean speed) for a period time, such as 30 seconds, which can be used as a known reference point for estimations. During the washout pulse movement of the rotor can be indicative of the filling volume and therefor used for absolute pressure estimation if suction is induced. Similarly, movement of the rotor in response to pulses can be used to estimate the lumped compliance of the system or suction.

Additionally, time domain processing examining rates of change, integrals, or maximum/minimum values over a pulse period, of the bearing and/or drive indicators can used to extract additional information.

In a further example, the controller could be configured to use vibration (axial or tilt) of the rotor to derive additional information. For example, in some patient conditions the vibration of the rotor could be larger than other conditions and the measurement of such a vibration parameter could provide new estimates or improve other estimates. For example when the systemic resistance is high (low pump flow) or systemic resistance is low (high pump flow) the rotor can be pushed radially by hydraulic forces within the casing causing the rotor to vibrate more than when the resistance is at a more normal range. In one example, the vibration of the rotor can be defined as the overall magnitude of the time varying movement of the rotor, but could alternatively be calculated using only the rotor movement at the rotational frequency harmonics or a specific subset of harmonics (i.e. magnitude of rotor axial movement at 40, 80, 120, and 400 Hz for a rotor that is spinning at 2400 RPM which has a fundamental rotational frequency of 40 Hz).

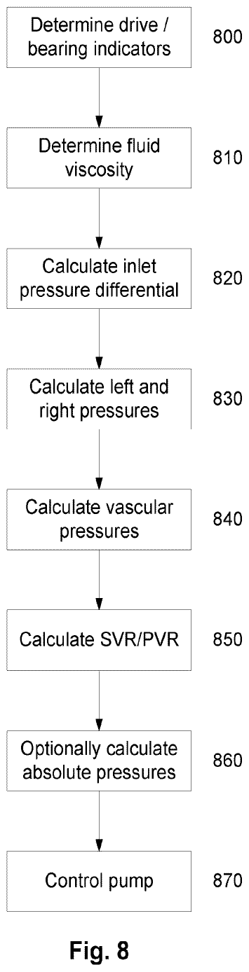

An example process for calculating specific hemodynamic parameters will now be described in more detail with reference to FIG. 8.

In this example, at step 800, the controller 150 operates to determine drive and bearing indicators, in a manner similar to that described above.

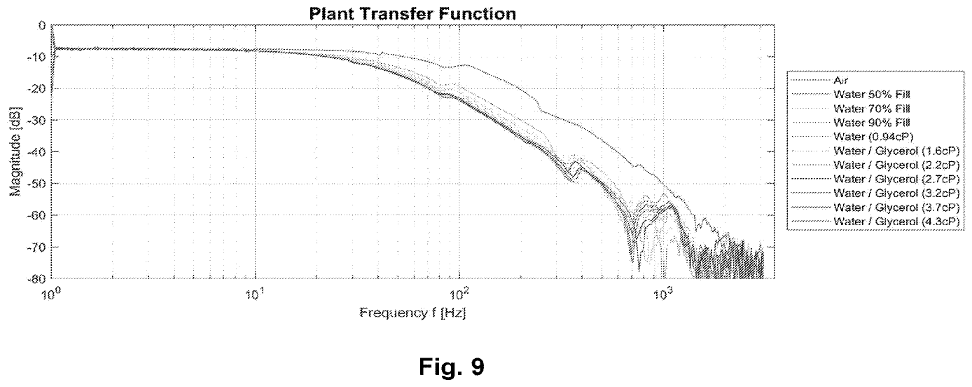

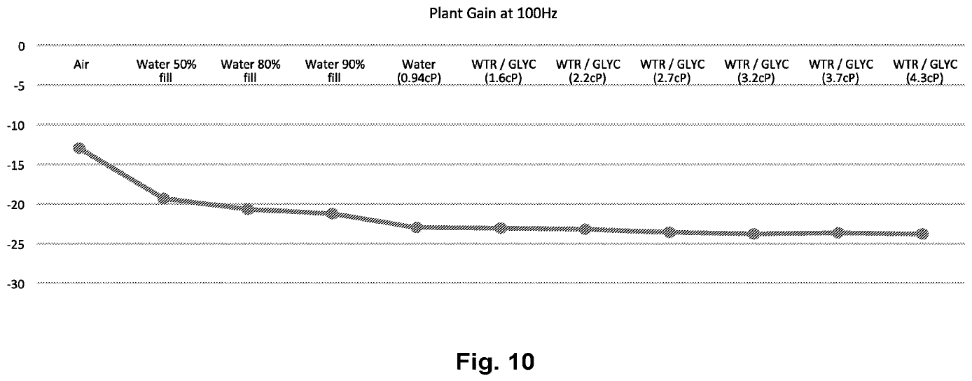

At step 810 fluid viscosity is determined, either by way of manual input of haematocrit, or through estimation approaches. Practically this can be achieved a few different ways, such as by looking at the plant transfer function, an example of which is shown in FIG. 9. In this regard, selection of a specific frequency (e.g. 100 Hz), allows the viscosity of the fluid being pumped to be estimated based on the plant gain, as shown in FIG. 10. This approach can be used for estimating haematocrit, which in turn can be used for patient diagnostics, as well as estimating the presence or amount of air in a pump during implant. In this regard, a presence or amount of air in the pump can be estimated using a plant transfer function, a fluid viscosity and/or variations in the bearing indicator. Additionally, and/or alternatively, viscosity can be determined by examining the location and/or magnitude of dynamic modes at higher frequencies.

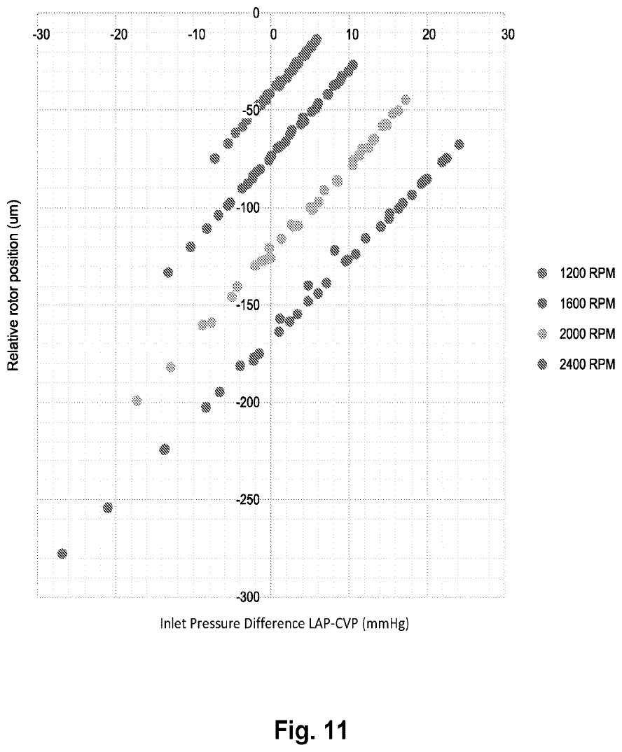

At step 820, the controller 150 operates to determine an inlet pressure differential. In this regard, FIG. 11 illustrates that there is a very strong correlation between the axial force, in one example derived from a rotor VZP (Virtual Zero Power) position and the relative inlet pressures. As such, for a dual sided pump, the relative inlet pressure of the left and right pump can be determined by observing the rotor VZP position, when controlling for other factors such as rotor speed, pulse height, hematocrit and bias current.

In this example the inlet pressure differential can be calculated using an equation of the following form, where equation constants are calculated experimentally:

Rotor position for LAP equal to RAP=β₁+β₂Nₙₒᵣₘ+β₃Nₙₒᵣₘ²+β₄Pₙₒᵣₘ+β₅PₙₒᵣₘNₙₒᵣₘ+β₆Hₙₒᵣₘ+β₇vₙₒᵣₘ+β₈dᵢₙₗₑₜ

$${N}_{norm} = Speed in k RPM [ k RPM ]$$

$${P}_{norm} = \frac{{P}_{motor}}{{( {N}_{kRPM} )}^{2}} = Speed normalized power [ \frac{W}{{k RPM}^{2}} ]$$

$${z}_{norm} = {z}_{abs} - {z}_{offset} = Position normalized [ um ]$$

$${H}_{norm} = pk - pk pulse height normalized [ k RPM ]$$

$${v}_{norm} = Hematocrit [ \% ]$$

$${d}_{inlet} = inlet pressure difference [ mm Hg ]$$

If a VZP controller is not used or different tuning in used then the MB current could also be used to estimate inlet pressure difference, using the tilt on the rotor (measured via the MB current differences), or used in conjunction with rotor position to determine the inlet pressure difference. In this instance, an equation of the following form can be used:

$$Inlet Pressure Difference = {β}_{1} + {β}_{2} {N}_{norm} + {β}_{3} {{N}_{norm}}^{2} + {β}_{4} {P}_{norm} + {β}_{5} {P}_{norm} {N}_{norm} + {β}_{6} {z}_{norm} + {β}_{7} {H}_{norm} + {β}_{8} {H}_{norm}^{2} + {β}_{9} {H}_{norm} {N}_{norm} + {β}_{10} {z}_{norm}^{2} + {β}_{11} {v}_{norm} + {β}_{12} {v}_{norm} {P}_{norm} + {β}_{13} {i}_{bias}$$

$${N}_{norm} = Speed in k RPM [ k RPM ]$$

$${P}_{norm} = \frac{{P}_{motor}}{{( {N}_{kRPM} )}^{2}} = Speed normalized power [ \frac{W}{{k RPM}^{2}} ]$$

$${z}_{norm} = {z}_{abs} - {z}_{offset} = Position normalized [ um ]$$

$${H}_{norm} = pk - pk pulse height normalized [ k RPM ]$$

$${v}_{norm} = Hematocrit [ \% ]$$

$${i}_{bias} = MB bias current [ A ]$$

Following this, left and right pump pressures can be calculated at step 830. Again, this can be achieved using equations of the following forms, with equation constants calculated experimentally:

$$Left pump pressure = {β}_{1} + {β}_{2} {N}_{norm} + {β}_{3} {{N}_{norm}}^{2} + {β}_{4} {P}_{norm} + {β}_{5} {P}_{norm} {N}_{norm} + {β}_{6} {z}_{norm} + {β}_{7} {H}_{norm} + {β}_{8} {H}_{norm}^{2} + {β}_{9} {H}_{norm} {N}_{norm} + {β}_{10} {z}_{norm}^{2} + {β}_{11} {v}_{norm} + {β}_{12} {v}_{norm} {P}_{norm} + {β}_{13} {i}_{bias}$$

$$right pump pressure = {β}_{1} + {β}_{2} {N}_{norm} + {β}_{3} {{N}_{norm}}^{2} + {β}_{4} {P}_{norm} + {β}_{5} {P}_{norm} {N}_{norm} + {β}_{6} {z}_{norm} + {β}_{7} {H}_{norm} + {β}_{8} {H}_{norm}^{2} + {β}_{9} {H}_{norm} {N}_{norm} + {β}_{10} {z}_{norm}^{2} + {β}_{11} {v}_{norm} + {β}_{12} {v}_{norm} {P}_{norm} + {β}_{13} {i}_{bias}$$

$${N}_{norm} = Speed in k RPM [ k RPM ]$$

Having established these values, further hemodynamic parameters including vascular pressures can now be calculated at step 840.

In this regard, it will be noted that whilst estimation of flow and pressure output of the LVAD is possible and established in the field, an LVAD is in parallel with left ventricle and in series with the right ventricle, so it is difficult to determine patient hemodynamics with only LVAD information.

A TAH with 2 independent pumps could estimate the flow and the pressure head of each pump, however without information about the relative pressures of the left and right pump it is not possible to know the individual Systemic Vascular Resistance (SVR) and Pulmonary Vascular Resistance (PVR) only the total vascular resistance (SVR+PVR).

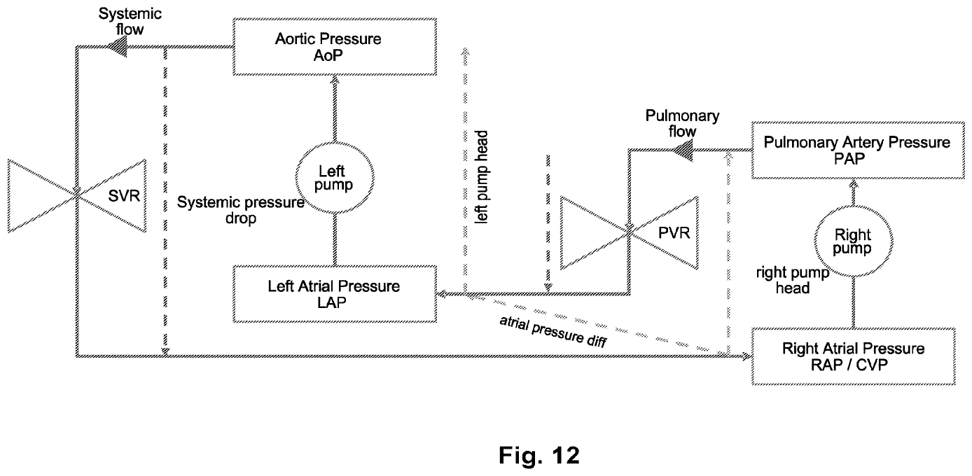

In the case of a dual sided pump, the relative left and right atrial filling can be used to estimate the SVR and PVR of the patient. In this regard, as shown in FIG. 12, knowing the left and right pump pressures, and the atrial pressure difference (given by the inlet pressure difference), allows the pulmonary and systemic pressure drops to be calculated. This in turn allows the SVR and PVR to be determined at step 850 as follows:

$$SVR = \frac{80 \times [ Systemic Pressure Drop ]}{Systemic Flow}$$

$$PVR = \frac{80 \times [ Pulmonary Pressure Drop ]}{Pulmonary Flow}$$

Furthermore, as all four of the relative inlet and outlet pressures can be estimated (Left in, Left out, Right in, Right out), then the absolute pressures can be determined at step 860 if one of the pressures is known. Clinically the easiest measurement would be central venous pressure (CVP), which would in turn allow calculation of left atrial pressure (LAP), pulmonary arterial pressure (PAP) and aortic pressure (AoP).

Additionally, a measure of delivered oxygen can be more clinically relevant than the pressures. Delivered oxygen (DO₂) can be calculated using haemoglobin (Hb)/HCT, oxygen saturation (SaO₂) and the oxygenation pressure in arterial blood (PaO₂), one or more of which can be estimated using the drive and/or bearing indicator, and/or measured using other means. For example, SaO₂ can be calculated from a pulse oximeter, which can be achieved using the pump described above operating in a pulsatile manner.

The amount of delivered oxygen is given by the equation, where CO is the cardiac output:

DO₂=CO[(Hb×SaO₂×1.34)+(PaO₂×0.0031)]

Again monitoring of delivered oxygen can be used to adjust pump operation, for example increasing/decreasing flow rates in the pulmonary or systemic circulatory systems.

Additionally, the oxygenation level of the blood can change its viscosity values at different frequencies, and so monitoring at different frequencies can be used to allow oxygenation levels to be determined.

It should be noted that in practice, the above calculations may need to be modified to account for bronchial shunt, which is a physiological phenomena in which lung tissue is perfused with oxygenated blood from the systemic circulation that is then returned in part to the pulmonary veins/left atrium, in contrast to typical flow patterns where the oxygenated blood from the aorta is returned to the right atrium. The bronchial shunt is another flow path that can be added into the above calculations, to thereby obtain more accurate pressure estimates.

In healthy patients the bronchial shunt is only 1% of the total blood flow, whereas in heart failure patients it can be as high as 4%. Due to the nature of the shunt path, variation in the shunt resistance/flow does not significantly affect the left estimate, but can alter the right estimate. To account for this, in one example, the above calculations can be modified to include an assumed level of bronchial shunt, or alternatively, additional parameters could be used to estimate the shunt flow.

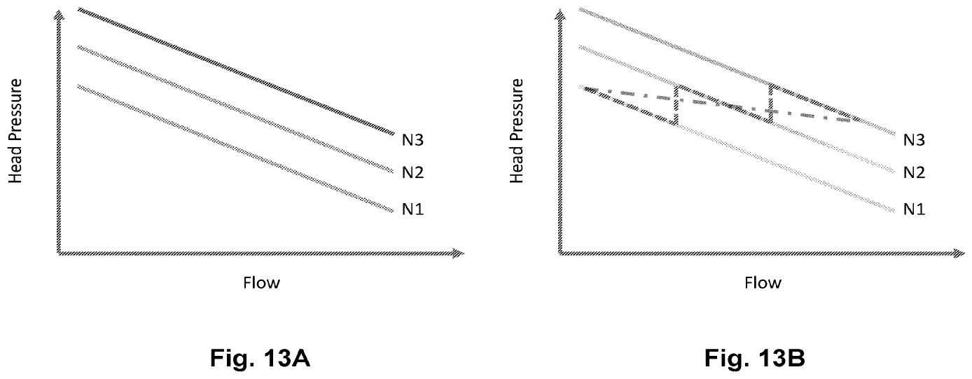

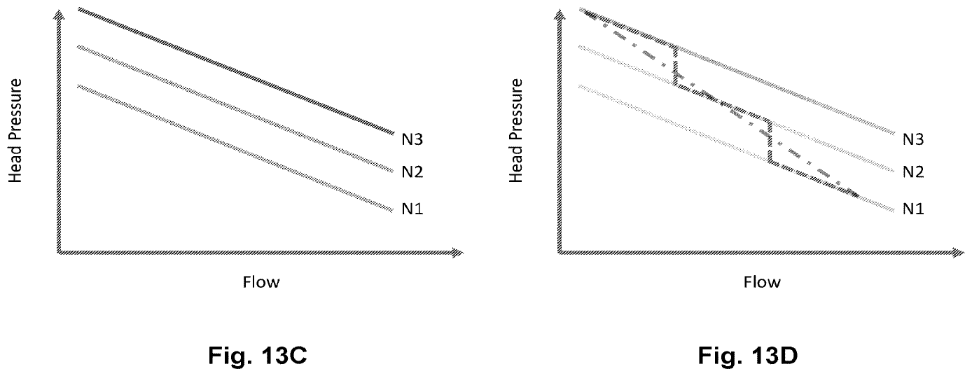

At step 870, the controller 150 operates to control the pump, based on the determined pressures. This can include adjusting the drive and/or bearing as required. In one example, with accurate estimation of the pump outflow, it is possible to change the pressure flow relationship of the pump by modifying the speed as a function of flow or pressure, which can in turn be used to make the HQ curve steep or shallow (see next slides), as shown for example in FIGS. 13A, 13B, 13C and 13D, respectively.

In this example, the controller is configured to predict head pressure, with an error being inputter as head pressure minus predicted head pressure. Proportional integral control is then used to control to a speed target, with autonomous changes to the speed would be limited to a defined safe range.

It will be appreciated that the above arrangement therefore uses a combination of drive and bearing signals to provide a more accurate estimate of hemodynamic parameters, which can be further improved through the inclusion of hematocrit as an indication of fluid viscosity. Furthermore, in the case of a dual sided impeller BiVAD, this can be used to resolve absolute pressures within a patient given a clinical measure of a single pressure.

The above described arrangement can be employed in wide range of circumstances and in different pump configurations. For example, this can be used when one or two pumps are used to provide assistance or replacement of the left or right ventricles, including in a TAH, when two rotary pumps to provide complete replacement of the native heart, in an LVAD/RVAD, when a single rotary pump is used to provide assistance to either the left or right ventricles, or in a BiVAD, when two rotary pumps to provide assistance to both the left or right ventricles.

The controller and control process can be used in a device that uses an active magnetic bearing in conjunction with a zero power controller that controls the position of the rotor in response to a change of magnetic bearing current. Signal filtering techniques on the fundamental current signal with consideration to the zero power controller can return a feedback signal appropriate for use in the controller.

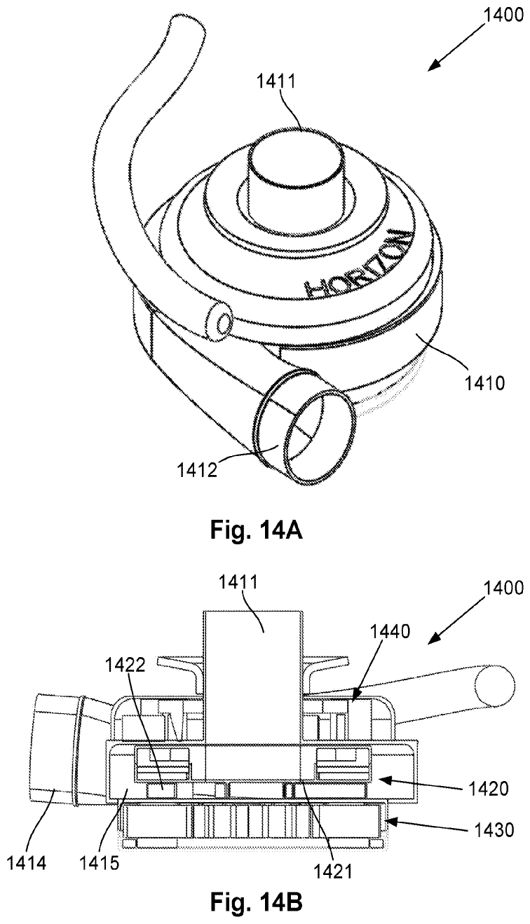

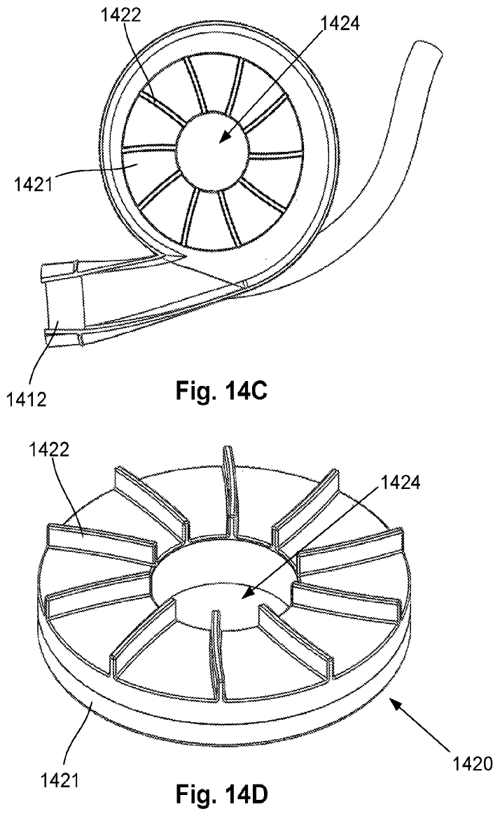

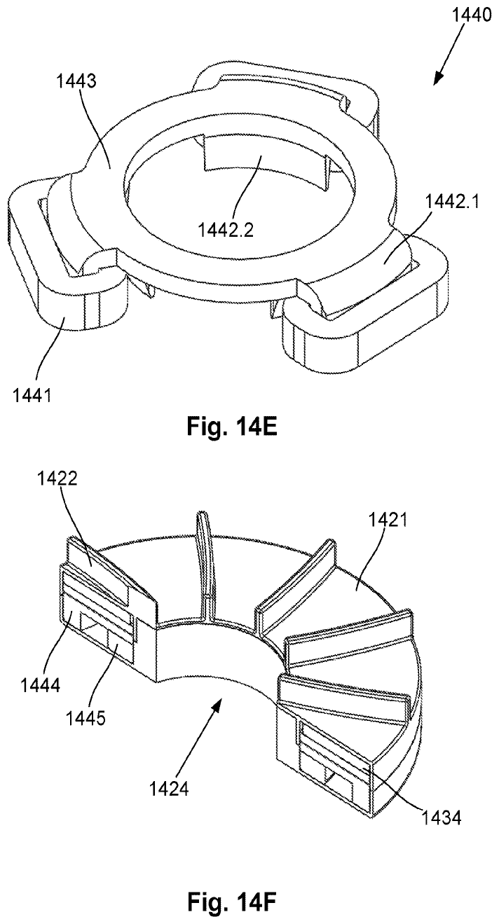

An example of a single VAD heart pump will now be described with reference to FIGS. 14A to 14F.

In this example, the heart pump 1400 includes a housing 1410 defining a cavity 1415. The housing can be of any suitable form but typically includes a main body, and left and right end caps which connect to the main body. The housing can be made of any suitable biocompatible material, and can be made of titanium, a polymer or the like.

The housing 1410 includes an inlet 1411, for connection to the left atrium/pulmonary vein or right atrium/vena cava, or left or right ventricle, and an outlet 1412 for connection to the aorta or pulmonary artery, respectively.

The heart pump 1400 includes an impeller 1420 provided within the cavity 1415. The impeller 1420 includes a rotor 1421 having vanes 1422 mounted thereon for urging fluid from the inlet 1411 to the outlet 1412 upon rotation of the impeller 1420. In this example, as the heart pump 1400 is a single ventricular assist device, the impeller includes a single set of vanes 1422 for urging fluid from the inlet 1411 to the outlet 1412. In this example, the vanes 1422 have a configuration similar to that described above, and these will not therefore be described in further detail, although it will be appreciated that other suitable vane configurations can be used. The impeller can also include an aperture 1424 extending therethrough to allow blood to flow around the rear surface of the impeller and thereby prevent stagnation and clotting of blood within the heart pump. Furthermore, the use of a magnetic bearing in this region allows for blood gaps in excess of 200-300 μm, which can both reduces shear stress and thus red cell lysis, as well as promote greater rates of washout flow than otherwise anticipated in gaps created by hydrodynamic bearings.

The heart pump 1400 further includes a drive 1430 that rotates the impeller 1420 within the cavity 1415. The drive 1430 can be of any appropriate form but typically includes a number of coils, each wound on a respective stator, supported by a mounting, allowing the drive 1430 to be coupled to the housing 1410. The drive cooperates with magnetic material 1434 mounted in the rotor 1421, with the magnetic material being in the form of a number of circumferentially spaced permanent drive magnets arranged proximate an outer circumferential edge of the rotor 1421. In one example, the coils and stators are wedge shaped and circumferentially spaced around the mounting, so as to provide twelve electromagnets radially aligned with the drive magnets 1434 in the rotor 1421, to thereby maximise a degree of magnetic coupling between the magnets in the rotor 1421 and the drive 1430.

The heart pump 1400 can further include a magnetic bearing 1440 including at least one bearing coil 1441 that controls an axial position of the impeller within the cavity 1415. In one particular example, shown in more detail in FIG. 14E, the magnetic bearing includes three bearing coils 1441, each of which is mounted on a first leg 1442.1 of respective U-shaped stators, with a second leg 1442.2 being positioned radially inwardly of the first leg 1442.1. The stators are mounted to or integrally formed with a support 1443 and circumferentially spaced 140° apart around the housing so that the first and second legs 1442.1 1442.2 align with respective magnetic material, such as bearing magnets 1444, 1445 within the impeller 1420, allowing an axial position of the impeller 1420 to be controlled.

In one particular example, the bearing rotor assembly includes ferromagnetic core target 1444 mounted in the rotor, proximate an outer circumferential edge of the rotor 1421, and a permanent bearing magnet or ferromagnetic material 1445 mounted radially inwardly of the first ferromagnetic core target 1444, so that the ferromagnetic core target and bearing magnets 1444, 1445 align with respective legs 1442.1, 1442.2 of the stators. The ferromagnetic core target can be replaced with a second permanent magnet. However, the use of a magnetic bearing may not be required and can be replaced by a static physical bearing or hydrodynamic bearing, or the like.

In this example, the drive 1430 and magnetic bearing 1440 are mounted at opposing ends of the housing 1410 so that the drive and bearing 1430, 1440 are provided proximate opposing surfaces of the rotor 1421 as shown for example in FIG. 14B. In the current example the drive 1430 is mounted adjacent the side of the impeller 1420 that includes vanes so as to maximise the blood gap between the rotor, vanes and the casing. That is to say, only the vane tips are in closer proximity to the casing, however this blood gap can still be in the order of 200-300 μm. Additionally, bearing and drive are configured so that the magnetic forces inherent between the drive 1430 and impeller 1420, and between the magnetic bearing 1440 and impeller 1420 and the hydraulic forces on the impeller 1420 define a balance position within the cavity under conditions of normal flow. This minimises the bearing current required to maintain the position of the impeller 1420 within the cavity under nominal flow conditions.

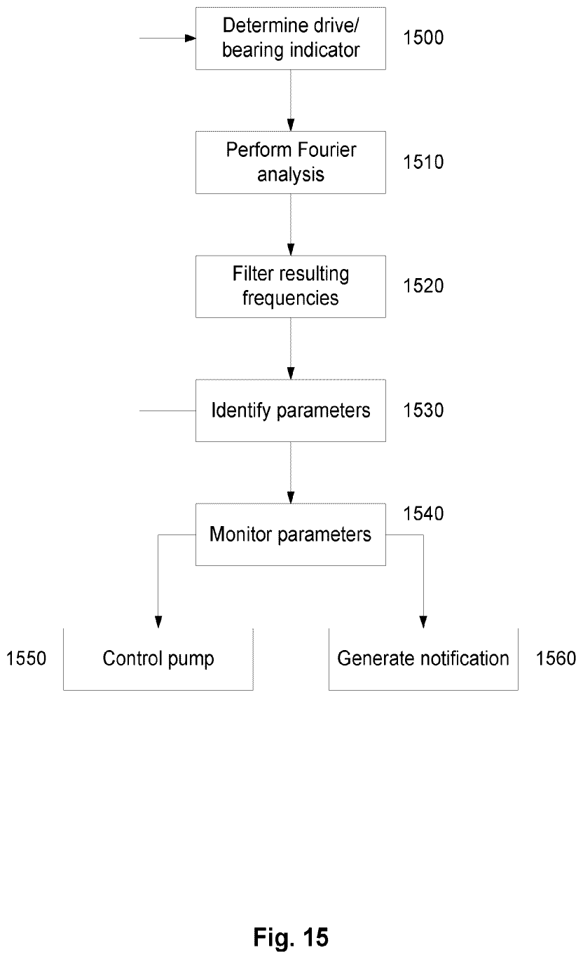

Periodic patient signals such as breathing rate, breathing depth, vascular compliance, atrial contractility and ventricular contractility (in the case of an LVAD) can be detected through the magnetic bearing and drive signals, including allowing both a rate and magnitude of signals to be estimated. An example of this process will now be described with reference to FIG. 15.

In this example, at step 1500, the controller 150 operates to determine drive and/or bearing indicators, in a manner similar to that described above.

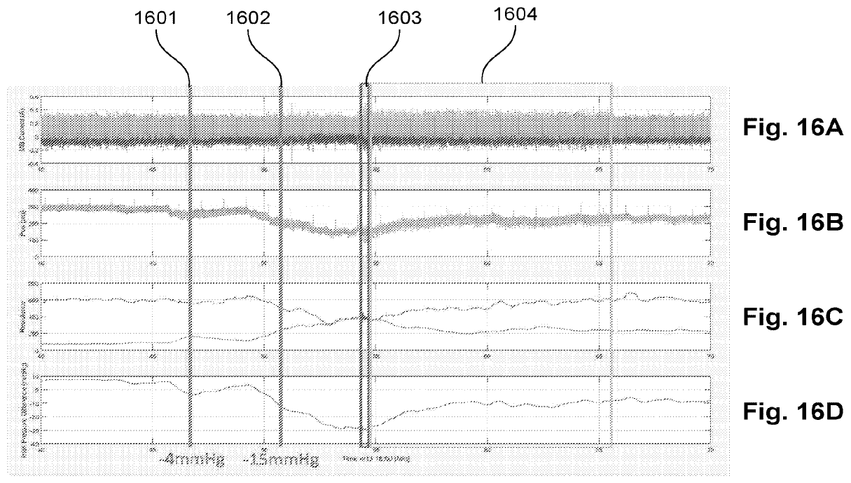

Examples of the magnetic bearing signals monitored during a monitoring period are shown in FIG. 16A, with a derived impeller position being shown in FIG. 16B. Measured vascular resistance and derived pump inlet pressure differences are shown in FIGS. 16C and 16D. In these examples, changes in inlet pressure are shown at 1601, 1602, whilst periods of increased pulse magnitude caused by pumps speeds of 600 RPM and 300 RPM (as opposed to the default 100 RPM), being shown at 1603, 1604.

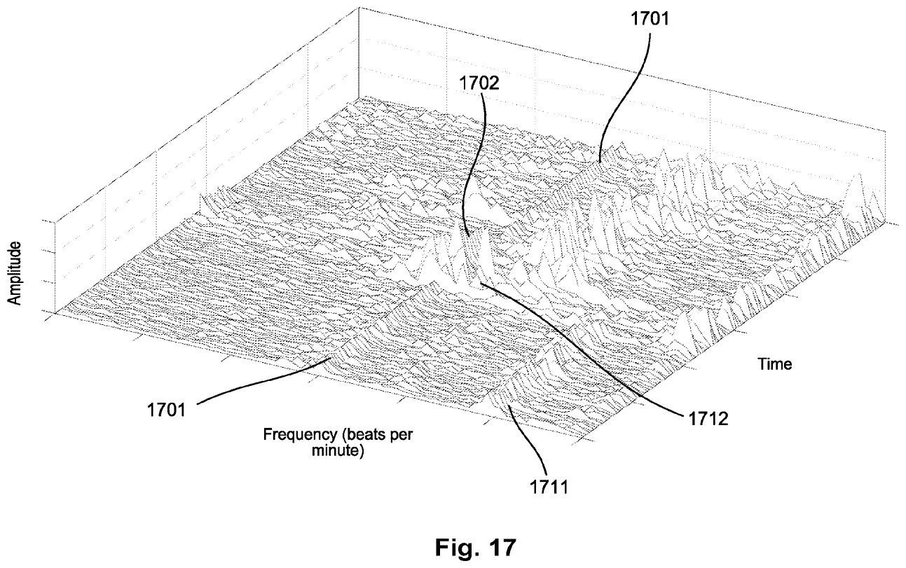

At step 1510 Fourier analysis is performed, to determine the magnitude of the signals at different frequencies. FIG. 17 is an example of a waterfall plot resulting from Fourier analysis performed using the bearing indicator signals of FIG. 16A.

The plot shows peaks corresponding to the heart pulsatile flow harmonics at 1701, 1702, with the increased magnitude peaks caused by the increased rotation speed pulse magnitude being shown at 1702. A normal breathing rate is shown by the peaks 1711, whereas elevated breathing rate and magnitude is shown at 1712. It will be appreciated that this therefore allows information regarding a breathing rate to be determined.

This can be facilitated at step 1520, for example by filtering out signals resulting from operation of the pump, thereby effectively removing the peaks 1701, 1702.

At step 1530, one or more periodic parameters can be extracted from the results of the Fourier analysis, with these being monitored at step 1540, for example comparing these to desirable or expected ranges, to ascertain when parameters fall out of range. This can then be used to control the pump and/or generate a notification at steps 1550 or 1560. For example, if a breathing rate is high, this could indicate reduced oxygenation, and so pump speed could be increased. Alternatively and/or additionally, an alert could be generated to notify medical personnel that the values are out of range and an intervention is required.

Thus, frequency domain analysis and filtering can be used to identify periodic subject signals, with breathing in particular being used to identify hazardous events, allowing clinical staff to be informed using alarms, messages, metrics, or the like, and also optionally allowing the pump operation to be controlled. Whilst this example has focused on use of the bearing indicator, as described above this could also or alternatively use the drive indicator.

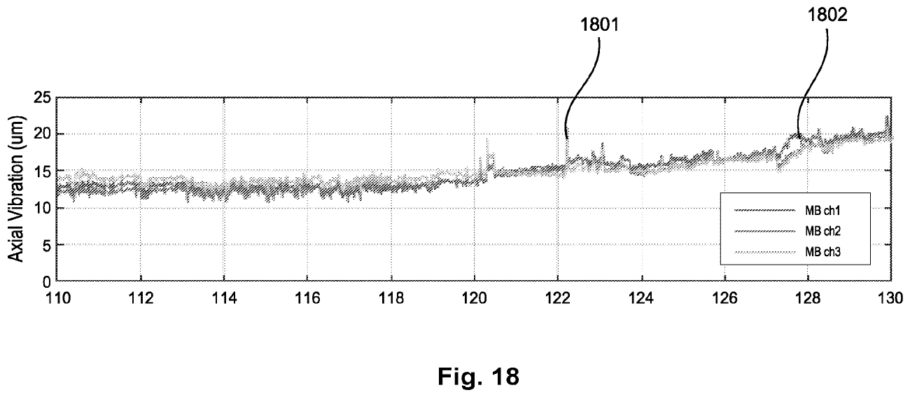

The above described approaches can also be used to measure other parameters, such pump parameters or pump operational performance, specifically if there are any clots, thrombus, or occlusions that are upstream, inside, or downstream to the device and its vascular connections. One example of this would be to monitor for vibration of the rotor and if the vibration magnitude increases that could indicate that there is a thrombus attached to the rotor which is changing the rotational balance of the spinning rotor. An example of this is shown in FIG. 18, in which there are increased peaks 1801 and a spread 1802 in different magnetic bearing signals, indicating the impeller is undergoing vibration, and hence indicating the presence of a thrombus.

Monitoring the magnetic bearing signal, and in particular monitoring changes in the bearing parameter to detect thrombus can also be used to determine a location of the thrombus, for example determining whether this is attached to the impeller, and/or partially blocking an inlet or outlet of the pump, which in turn can be used to determine the best intervention for treatment.

In one example, the above described arrangement uses a bearing indicator to calculate one or more of the following parameters: fluid viscosity, a presence of a thrombus or occlusion, location of a thrombus or occlusion, a breathing rate, a breathing depth, a vascular compliance (inflow or outflow), a ventricular contractility magnitude, an atrial contractility magnitude, an atrial contractility rate, a patient activity, such as running, sleeping, resting or the like, or postural changes.

In one example, the above described arrangement uses both drive and bearing indicators to calculate one or more of the following parameters: a breathing rate, a breathing depth, a flow, a head pressure, a relative inlet pressure, an absolute pressure within a subject, a systemic/pulmonary vascular pressure, a systemic/pulmonary vascular resistance, a vascular compliance (inflow or outflow), a ventricular contractility magnitude, an atrial contractility magnitude, a level of delivered oxygen, specific events within cardiac cycle, such as aortic valve opening/closing, mitral valve opening/closing, a ratio of systemic and pulmonary vascular resistance, a ventricular contractility rate, an atrial contractility rate, a patient activity, such as running, sleeping, resting or the like, or postural changes.

In one example, the above described arrangement uses a drive bearing indicator to calculate one or more of the following parameters: a breathing rate, a breathing depth, a vascular compliance (inflow or outflow), an atrial contractility magnitude, or an atrial contractility rate.

Whilst the above parameters might be derivable for both total artificial hearts and ventricular assist devices, it will be noted that the configuration of the pump described in FIGS. 1 to 3, which uses a single dual sided impeller to provide pulmonary and systemic flows is particularly beneficial for deriving hemodynamic parameters, such as a flow, a head pressure, a relative inlet pressure, an absolute pressure within a subject, a systemic/pulmonary vascular pressure, a systemic/pulmonary vascular resistance, a ratio of systemic and pulmonary vascular resistance, or a patient activity.

In connection with techniques described herein, it will be appreciated that the location/placement of the heart pump may make obtaining data regarding operation of the heart pump and the subject (including hemodynamic attributes of the subject) more technically challenging in some instances (e.g., because it is located inside the body of an individual). Accordingly, the ability to calculate one or more of the parameters described herein can be advantageous to providing improved therapy/therapeutic treatment for the subject. For example, by increasing the efficiency at which the heart pump operates. Or providing dynamic feedback to the individual or clinician. Or automatically controlling aspects of the heart pump based on one or more parameters. Thus, the techniques herein for calculating parameter(s) can be used to improve the health, well-being, and the like of individuals using heart pumps. The techniques can be used by clinicians to provide increased quality of care that is responsive or based on how the calculated parameter(s) change. The approach can also avoid the need to implant additional separate sensors, such as blood pressure sensors, or the like.

It will be appreciated as in the previous example, the apparatus can further include a controller, and otherwise functions largely as previously described, and hence will not be described in further detail.

Throughout this specification and claims which follow, unless the context requires otherwise, the word “comprise”, and variations such as “comprises” or “comprising”, will be understood to imply the inclusion of a stated integer or group of integers or steps but not the exclusion of any other integer or group of integers. As used herein and unless otherwise stated, the term “approximately” means ±20%. The above embodiments are to be understood as non-limiting illustrative examples of how the present invention, and aspects of the present invention, may be implemented. Further examples of the present invention are envisaged such as applying the invention to axial impellers including axial flow pumps. It is to be understood that any feature described in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the present invention, which is defined in the accompanying claims.

It is to be noted that the term “or” as used herein is to be interpreted to mean “and/or”, unless expressly stated otherwise. Also, the phrase “at least one” means at least one of the elements listed rather than at least one of each of the elements listed, unless expressly stated otherwise.

1. A heart pump including:
a) a housing forming a cavity including at least one inlet and at least one outlet;
b) an impeller provided within the cavity, the impeller including vanes for urging fluid from the inlet to the outlet upon rotation of the impeller;
c) a drive that rotates the impeller within the cavity;
d) a magnetic bearing including at least one bearing coil that controls an axial position of the impeller within the cavity; and,
e) one or more electronic processing devices that are configured to:
i) determine a bearing indicator relating to axial forces on the impeller; and,
ii) use the bearing indicator to calculate at least one parameter.

2. A heart pump according to claim 1, wherein the bearing indicator is indicative of at least one of:
a) a tilt of the impeller;
b) an electrical current used by the magnetic bearing;
c) an axial position of the impeller within the cavity;
d) a change in tilt of the impeller;
e) a rate of change in tilt of the impeller;
f) a change in bearing indicator;
g) a rate of change in bearing indicator;
h) a derivative of bearing indicator;
i) an integral of bearing indicator;
j) a magnitude in difference between maximum and minimum values of bearing indicator over a pulse period;
k) a change in electrical current used by the magnetic bearing;
l) a rate of change in electrical current used by the magnetic bearing;
m) a derivative of electrical current used by the magnetic bearing;
n) an integral of electrical current used by the magnetic bearing;
o) a magnitude in difference between maximum and minimum values of electrical current used by the magnetic bearing over a pulse period;
p) a change in axial position of the impeller within the cavity;
q) a measure of vibration of the impeller; and,
r) a rate of change in axial position of the impeller within the cavity.

3. A heart pump according to claim 1, wherein the one or more processing devices are configured to determine the bearing indicator based on at least one of:
a) signals from a position sensor configured to detect an axial position of the impeller;
b) signals from a position sensor configured to detect a radial position of the impeller; and,
c) a current supplied to the at least one bearing coil.

4. A heart pump according to claim 1, wherein the one or more electronic processing devices are configured to:
a) determine a drive indicator indicative of rotation of the impeller; and,
b) use the drive indicator and the bearing indicator to calculate the at least one parameter.

5. A heart pump according to claim 4, wherein the drive indicator is indicative of at least one of:
a) a current supplied to the drive;
b) an expected rotational speed of the impeller;
c) an actual rotational speed of the impeller;
d) a magnitude of a rotational speed change of the impeller;
e) a waveform of changes in rotational speed of the of the impeller; and,
f) a ratio of high speed and low speed periods of the waveform.

6. A heart pump according to claim 4, wherein the one or more processing devices are configured to determine the drive indicator based on a magnitude of current signals applied to the drive.

7. A heart pump according to claim 4, wherein the one or more processing devices are configured to:
a) determine a fluid viscosity indicator indicative of the viscosity of blood within the heart pump; and,
b) calculate at least one hemodynamic parameter using the drive indicator, the bearing indicator, and the fluid viscosity indicator.

8. (canceled)

9. (canceled)

10. (canceled)

11. A heart pump according to claim 1, wherein the parameter includes at least one of:
a) a subject parameter;
b) a pump operating parameter; and,
c) a hemodynamic parameter.

12. A heart pump according to claim 11, wherein the subject parameter includes at least one of:
a) a periodic subject signal;
b) a presence of a thrombus or occlusion;
c) a location of a thrombus or occlusion;
d) a breathing rate;
e) a breathing depth;
f) a subject activity level;
g) a subject posture or subject posture change; and,
h) at least one hemodynamic parameter, the at least one hemodynamic parameter including at least one of:
i) a flow;
ii) a head pressure;
iii) a relative inlet pressure;
iv) one or more absolute pressures within a subject;
v) a systemic vascular pressure;
vi) a pulmonary vascular pressure;
vii) a systemic vascular resistance;
viii) a pulmonary vascular resistance;
ix) a ratio of systemic and pulmonary vascular resistance;
x) vascular compliance;
xi) a ventricular contractility magnitude;
xii) a ventricular contractility rate;
xiii) an atrial contractility magnitude;
xiv) an atrial contractility rate;
XV) a level of delivered oxygen;
xvi) a cardiac event;
xvii) aortic valve opening or closing; and,
xviii) mitral valve opening or closing.

13. A heart pump according to claim 1, wherein the one or more processing devices are configured to:
a) determine at least one clinically measured pressure parameter; and,
b) calculate remaining absolute pressure parameters, wherein the pressure parameters include:
i) central venous pressure (CVP);
ii) left atrial pressure (LAP);
iii) pulmonary arterial pressure (PAP); and,
iv) aortic pressure (AoP).

14. A heart pump according to claim 1, wherein the one or more processing devices are configured to at least one of:
a) control the heart pump at least in part using the at least one parameter;
b) display an indication of the at least one parameter;
c) record, to non-transitory memory, an indication of the at least one parameter; and,
d) generate an alert or notification based on at least one parameter.

15. A heart pump according to claim 14, wherein the one or more processing devices are configured to control at least one of a rotational speed and an axial position of the impeller.

16. A heart pump according to claim 1, wherein the one or more processing devices are configured to control a rotational speed of the impeller to at least one of:
a) control a blood flow rate through the heart pump;
b) vary the rotational speed to induce pulsatile flow;
c) vary the rotational speed to induce pulsatile flow that mimics a physiological pulse;
d) vary the rotational speed to induce pulsatile flow that matches a physiological pulse when the heart pump is used as a ventricular assist device;
e) vary the rotational speed to induce pulsatile flow in accordance with a pulse waveform;
f) vary the rotational speed to alter a pulse waveform of pulsatile flow;
g) vary the rotational speed to alter a pulse waveform to at least one of:
i) alter a pulsatile frequency;
ii) alter a pulsatile magnitude; and,
iii) change a ratio of higher speed and lower speed parts of the pulse waveform;
h) vary the rotational speed to selectively load a subject's heart when the heart pump is used as a ventricular assist device;
i) vary the rotational speed to selectively load a subject's heart when the heart pump is used as a ventricular assist device to thereby promote recovery of the subject's heart;
j) increase an effective pump flow curve gradient; and,
k) decrease an effective pump flow curve gradient.

17. A heart pump according to claim 1, wherein the one or more processing devices are configured to one of:
a) perform spectral analysis of at least one of a drive indicator and the bearing indicator; and, determine at least one subject parameter using results of the performed spectral analysis;
b) determine a magnitude and/or frequency of periodic signals within at least one of a drive indicator and the bearing indicator; and, determine the parameter using the magnitude and/or frequency; and,
c) determine a waveform shape of signals within at least one of a drive indicator and the bearing indicator; and, determine the parameter using the waveform shape.

18. (canceled)

19. (canceled)

20. (canceled)

21. (canceled)

22. (canceled)

23. A heart pump according to claim 1, wherein the impeller includes first and second sets of vanes provided on a rotor body, the rotor being positioned within the cavity to define:
a) a first cavity portion having a first inlet and a first outlet, the first set of vanes being provided within the first cavity portion so as to define a first pump that provides at least partial left ventricular function; and,
b) a second cavity portion having a second inlet and a second outlet, the second set of vanes being provided within the second cavity portion so as to define a second pump that provides at least partial right ventricular function.

24. (canceled)

25. (canceled)

26. A heart pump according to claim 1, wherein the drive is positioned at a first end of the cavity and the magnetic bearing is positioned at a second end of the cavity and wherein:
a) the drive includes:
i) a number of circumferentially spaced permanent magnets mounted in the rotor of the impeller, adjacent magnets having opposing polarities; and,
ii) at least one drive coil that in use generates a magnetic field that cooperates with the magnetic material allowing the impeller to be rotated; and,
b) the magnetic bearing includes:
i) first and second annular magnetic bearing members mounted within and proximate a face of the rotor, the first magnetic bearing member being provided radially outwardly of the second magnetic bearing member;
ii) a number of circumferentially spaced substantially U-shaped bearing stators mounted in the housing proximate a second end of the cavity, each U-shaped bearing stator having first and second bearing stator legs substantially radially aligned with the first and second magnetic bearing members respectively; and,
iii) at least one bearing coil on each bearing stator that generates a magnetic field that cooperates with the magnetic bearing members to thereby at least one of:
(1) control an axial position of the impeller; and,
(2) at least partially restrain radial movement of the impeller.

27. (canceled)

28. (canceled)

29. (canceled)

30. (canceled)

31. A controller for a heart pump, the controller being configured to calculate at least one hemodynamic parameter, wherein the heart pump includes:
a) a housing forming a cavity including at least one inlet and at least one outlet;
b) an impeller provided within the cavity, the impeller including vanes for urging fluid from the inlet to the outlet upon rotation of the impeller;
c) a drive that rotates the impeller within the cavity;
d) a magnetic bearing including at least one bearing coil that controls an axial position of the impeller within the cavity; and,
e) the controller including one or more electronic processing devices that are configured to:
i) determine a bearing indicator relating to axial forces on the impeller; and,
ii) use the bearing indicator to calculate at least one parameter.

32. A method of calculating at least one parameter, wherein the method includes in one or more processing devices:
a) determining a bearing indicator associated with a magnetic bearing including at least one bearing coil that controls an axial position of an impeller within a cavity of a heart pump, wherein the bearing indicator relates to axial forces on the impeller; and,
b) using the bearing indicator to calculate at least one parameter.

33. A heart pump including:
a) a housing forming a cavity including at least one inlet and at least one outlet;
b) an impeller provided within the cavity, the impeller including vanes for urging fluid from the inlet to the outlet upon rotation of the impeller;
c) a drive that rotates the impeller within the cavity;
d) a magnetic bearing including at least one bearing coil that controls an axial position of the impeller within the cavity; and,
e) one or more electronic processing devices that are configured to:
i) determine a drive indicator indicative of rotation of the impeller; and,
ii) use the drive indicator to calculate the at least one parameter.

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)
